SEQUENCE LISTING (15 sequences)

SEQ ID NO: 1 - DNA, 25 bp, Artificial Sequence
  Synthesized Peptide
uggcgcuuucuacaaggcauuugua

SEQ ID NO: 2 - DNA, 25 bp, Artificial Sequence
  synthesized peptides
ugguuuccaucggaauuacugcgua

SEQ ID NO: 3 - DNA, 23 bp, Artificial Sequence
  qRT-PCR
ttcttttcagtgttgtgaccaag

SEQ ID NO: 4 - DNA, 19 bp, Artificial Sequence
  Primers
gccccgaatgtggtaagtt

SEQ ID NO: 5 - DNA, 20 bp, Artificial Sequence
  Primers
ggcagtaaaccaggccaata

SEQ ID NO: 6 - DNA, 20 bp, Artificial Sequence
  Primer
agcagagccgtcacattctt

SEQ ID NO: 7 - DNA, 20 bp, Artificial Sequence
  Primer
ctatgagacggtgacgcaga

SEQ ID NO: 8 - DNA, 20 bp, Artificial Sequence
  Primer
cgggaggagacaaagtggta

SEQ ID NO: 9 - DNA, 21 bp, Artificial Sequence
  Primer
aaggacgacggcaactacaag

SEQ ID NO: 10 - DNA, 21 bp, Artificial Sequence
  Primer
atgccgttcttctgcttgtcg

SEQ ID NO: 11 - DNA, 23 bp, Artificial Sequence
  Primer
tgtccacaccatctaagactgaa

SEQ ID NO: 12 - DNA, 24 bp, Artificial Sequence
  primer
aggattttagtttccacattttgc

SEQ ID NO: 13 - DNA, 18 bp, Artificial Sequence
  Primer
gcactcttccagccttcc

SEQ ID NO: 14 - DNA, 15 bp, Artificial Sequence
  Primer
ccgctcaggagcaat

SEQ ID NO: 15 - protein, 1070 aa, Homo sapiens
MCFSFIMPPAMADILDIWAVDSQIASDGSIPVDFLLPTGIYIQLEVPREATISYIKQMLWKQVHNYPMFNLLMDIDSYMFACVNQTAVYEELEDETRRLCDVRPFLPVLKLVTRSCDPGEKLDSKIGVLIGKGLHEFDSLKDPEVNEFRRKMRKFSEEKILSLVGLSWMDWLKQTYPPEHEPSIPENLEDKLYGGKLIVAVHFENCQDVFSFQVSPNMNPIKVNELAIQKRLTIHGKEDEVSPYDYVLQVSGRVEYVFGDHPLIQFQYIRNCVMNRALPHFILVECCKIKKMYEQEMIAIEAAINRNSSNLPLPLPPKKTRIISHVWENNNPFQIVLVKGNKLNTEETVKVHVRAGLFHGTELLCKTIVSSEVSGKNDHIWNEPLEFDINICDLPRMARLCFAVYAVLDKVKTKKSTKTINPSKYQTIRKAGKVHYPVAWVNTMVFDFKGQLRTGDIILHSWSSFPDELEEMLNPMGTVQTNPYTENATALHVKFPENKKQPYYYPPFDKIIEKAAEIASSDSANVSSRGGKKFLPVLKEILDRDPLSQLCENEMDLIWTLRQDCREIFPQSLPKLLLSIKWNKLEDVAQLQALLQIWPKLPPREALELLDFNYPDQYVREYAVGCLRQMSDEELSQYLLQLVQVLKYEPFLDCALSRFLLERALGNRRIGQFLFWHLRSEVHIPAVSVQFGVILEAYCRGSVGHMKVLSKQVEALNKLKTLNSLIKLNAVKLNRAKGKEAMHTCLKQSAYREALSDLQSPLNPCVILSELYVEKCKYMDSKMKPLWLVYNNKVFGEDSVGVIFKNGDDLRQDMLTLQMLRLMDLLWKEAGLDLRMLPYGCLATGDRSGLIEVVSTSETIADIQLNSSNVAAAAAFNKDALLNWLKEYNSGDDLDRAIEEFTLSCAGYCVASYVLGIGDRHSDNIMVKKTGQLFHIDFGHILGNFKSKFGIKRERVPFILTYDFIHVIQQGKTGNTEKFGRFRQCCEDAYLILRRHGNLFITLFALMLTAGLPELTSVKDIQYLKDSLALGKSEEEALKQFKQKFDEALRESWTTKVNWMAHTVRKDYRS

(19) United States
(12) Patent Application Publication
(10) Pub. No.: US 2012/0231015 A1
(43) Pub. Date: Sep. 13, 2012

(54) FRAGILE X MENTAL RETARDATION PROTEIN (FMRP), COMPOSITIONS, AND METHODS RELATED THERETO

(75) Inventors: Gary Bassell, Snellville, GA (US); Stephen Warren, Sandy Spring, GA (US); Christina Gross, Decatur, GA (US)

(73) Assignee: EMORY UNIVERSITY, Atlanta, GA (US)

(21) Appl. No.: 13/505,913

(22) PCT Filed: Nov. 4, 2010
(86) PCT No.: PCT/US10/55387

§ 371 Date: May 24, 2012

Related U.S. Application Data
(60) Provisional application No. 61/258,829, filed on Nov. 6, 2009.
(60) Provisional application No. 61/366,354, filed on Jul. 21, 2010.

(51) Int. Cl. A61K 31/713; G01N 33/573; A61K 31/352; A61K 31/7088; A61K 39/395; A61K 31/5377; G01N 27/62; A61P 25/00

(52) U.S. Cl. 424/158.1; 435/15; 435/7.4; 514/44 A; 514/44 R; 514/235.5; 514/453

(57) ABSTRACT

The invention relates to fragile X mental retardation protein (FMKP), compositions, and methods related thereto. In certain embodiments, the invention relates to treating a neurological disorder by administering a P13K antagonist to a subject in need thereof. In other embodiments, the invention relates to methods of diagnosing neurological disorders.

### FIELD

The invention relates to fragile X mental retardation protein (FMRP), compositions, and methods related thereto. In certain embodiments, the invention relates to treating a neurological disorder by administering a PI3K antagonist to a subject in need thereof. In other embodiments, the invention relates to methods of diagnosing neurological disorders, and monitoring the effect of a drug or therapy on such disorders by measuring PI3K.

### BACKGROUND

Fragile X syndrome (FXS) is the most frequent inherited form of intellectual disability. It is caused by loss of function of fragile X mental retardation protein (FMRP), an mRNA binding protein involved in the regulation of target mRNAs. FMRP causes dysregulation of basal and neurotransmitter-induced protein synthesis. The molecular mechanism whereby FMRP couples the activation of cell surface receptors to protein synthesis regulation is unclear. Several studies have shown exaggerated gp1 mGluR signaling in FXS animal models. The mGluR theory of FXS postulates that excessive signaling through gp1 mGluRs underlies synaptic defects observed in the absence of FMRP. However, no studies reveal how FMRP limits gp1 mGluR-induced signal transduction. Other signal transduction pathways besides gp1 mGluR signaling have been reported to be dysregulated in the absence of FMRP. Thus, it would be beneficial to identify how FMRP regulates signaling of gp1 mGluRs to identity improved therapeutic strategies to treat FMRP and related ASDs.

The PI3K kinase superfamily includes a large number of structurally related enzymes with differing regulation and substrates. Human PI3K comprises a regulatory subunit and a 110-kDa catalytic subunit (p110). PI3K acts, for example, through a downstream protein kinase B (PKB, also named Akt) to regulate many cellular processes including cell survival, cell proliferation, vesicular trafficking, inflammation, and apoptosis inhibition. When activated, PI3K phosphorylates phosphoinositides at the 3′ position of the inositol ring. Following their phosphorylation, the phosphoinositides promote activation of downstream signaling molecules, e.g. Akt and PDK1.

Hayashi et al. disclosed that the inhibition of p21-activated kinase (PAK) rescues symptoms of fragile X syndrome in mice. Proc Natl Acad Sci USA. 2007; 104(27):11489-94. US Patent App. Pub. 2010/0247552 provides that PAK is activated by PI3K signaling via PDK. See also US Patent App. Pub. 2009/0297573. US Patent App. Pub. 2009/0099077 provides that increasing PI3K levels may offset memory loss due to cognitive decline or due to neurodegenerative disorders. Methods for treating CNS disorders such as schizophrenia, psychosis and cognitive disorders using specific inhibitors of phosphatidylinositol-3-kinase p110 delta are disclosed in international PCT App WO/2010/065923. The preceding is not an admission that any of the references cited above are prior art.

### SUMMARY

It has been discovered that FMRP regulates the synthesis and synaptic localization of p110beta, the catalytic subunit of PI3K suggesting that dysregulated PI3K signaling may underlie the synaptic impairments in FXS. PI3K antagonists rescue FXS-associated phenotypes such as dysregulated synaptic protein synthesis, excess AMPA receptor internalization, and increased spine density.

In certain embodiments, the invention relates to methods of treating or preventing a disease related to fragile X mental retardation protein (FMRP) comprising administering a PI3K antagonist to a subject at risk of, exhibiting symptoms of, or diagnosed with the disease.

In certain embodiments, the invention relates to methods of treating synapse defects in the brain comprising administering a PI3K antagonist to a subject. In a typically embodiment, the subject is diagnosed with fragile X syndrome, autism, or an austism spectrum disorders (ASDs) such as Asperger syndrome, pervasive developmental disorder, atypical autism, childhood disintegrative disorder, or Rett syndrome.

In further embodiments, the PI3K antagonist is a broad spectrum PI3K antagonist. In further embodiments, PI3K antagonist preferentially binds p85 or p110 subunits such as the p110beta subunit. A compound that is a selective p110beta antagonist is 7-methyl-2-morpholino-9-(1-(phenylamino)ethyl)-4H-pyrido[1,2-a]pyrimidin-4-one. In further embodiments the PI3K antagonist is a short interfering nucleic acid (siNA) molecule of PI3K such as siRNA of p110alpha, p110beta, p110gamma or p110delta. In certain embodiments, the PI3K antagonist is an antibody to PI3K such as an antibody to p110alpha, p110beta, p110gamma or p110delta subunit. In certain embodiments, the PI3K antagonist is an aptamer to PI3K or p110alpha, p110beta, p110gamma or p110delta subunit.

In certain embodiments, the PI3K antagonist is wortmannin, 2-morpholin-4-yl-8-phenylchromen-4-one, SF1126 (RGD-conjugated prodrug of morpholin-4-yl-8-phenylchromen-4-one), 4-(2-(1H-indazol-4-yl)-6-(4-(methylsulfonyl)piperazin-1-yl)methyl)thieno[3,2-d]pyrimidin-4-yl)morpholine (GDC-0941), N-(7,8-dimethoxy-2,3-dihydroimidazo[1,2-c]quinazolin-5-yl)nicotinamide (PIK-90), (R)-2-(1-(7-methyl-2-morpholino-4-oxo-4H-pyrido[1,2-a]pyrimidin-9-yl)ethylamino)benzoic acid, PF-04691502, acetic acid (1S,4E,10R,11R,13S,14R)-[4-diallylaminomethylene-6-hydroxy-1-methoxymethyl-10,13-dimethyl-3,7,17-trioxo-1,3,4,7,10,11,12,13,14,15,16,17-do dec ahydro-2-oxa-cyclopenta[α]phenanthren-11-yl ester (PX-866), N-(3-(benzo[c][1,2,5]thiadiazol-5-ylamino)quinoxalin-2-yl)-4-methylbenzenesulfonamide (XL147), XL765, 2-((6-amino-9H-purin-9-yl)methyl)-5-methyl-3-(o-tolyl)quinazolin-4(3H)-one (CAL-101), BKM120, 2-methyl-2-(4-(3-methyl-2-oxo-8-(quinolin-3-yl)-2,3-dihydro-1H-imidazo[4,5-c]quinolin-1-yl)phenyl)propanenitrile (BEZ235), 7-methyl-2-(4-morpholinyl)-9-[1-(phenylamino)ethyl]-4H-pyrido[1,2-a]pyrimidin-4-one (TGX-221), TGX-155, 3-[4-(4-morpholinyl)pyrido[3′,′:4,5]furo[3,2-d]pyrimidin-2-yl]-phenol (PI-103), 5-[5-(4-fluoro-2-hydroxy-phenyl)-furan-2-ylmethylene]-thiazolidine-2,4-dione (AS-252424), 5-(6-quinoxalinylmethylene)-2,4-thiazolidinedione, 5-[(2,2-difluoro-1,3-benzodioxol-5-yl)methylene]-2,4-thiazolidinedione, (Z)-5-((4-(pyridin-4-yl)quinolin-6-yl)methylene)thiazolidine-2,4-dione (GSK1059615), 3,3′-(2,4-diaminopteridine-6,7-diyl)diphenol, or BGT226 or salt or prodrug thereof.

In certain embodiments, the invention relates to methods of diagnosing a disease related to fragile X mental retardation protein (FMRP) or PI3K signaling comprising assaying a sample from a subject for excessive PI3K or PI3K activity and correlating excessive PI3K or PI3K activity to a disease related to FMRP such as fragile X, autism, or ADSs.

In further embodiments, the assaying comprises detecting PI3K, e.g., p85alpha, p85beta, p110alpha, p110beta, p110gamma, or p110delta subunit and comparing the detected amount to that typically found in a sample from a person with or without a disease related to fragile X mental retardation protein. In certain embodiments, the PI3K, e.g., p85alpha, p85beta, p110alpha, p110beta, p110gamma, or p110delta subunit are detected by mass spectroscopy.

In further embodiments, the assaying comprising detecting phosphatidylinositol 3-phosphate in the sample and comparing the detected amount to that typically found in a sample from a person with or without a disease related to fragile X mental retardation protein, or with a disease and undergoing treatment. In certain embodiments, phosphatidylinositol 3-phosphate is detected by mass spectroscopy.

In certain embodiments, the assaying comprises isolating PI3K, e.g., p85alpha, p85beta, p110alpha, p110beta, p110gamma, or p110delta subunit from the sample providing isolates and measuring PI3K activity in the isolates.

In certain embodiments, the assaying comprises, combining the sample and affinity markers for PI3K, e.g., p85alpha, p85beta, p110alpha, p110beta, p110gamma, or p110delta subunit and measuring markers in the marker bound sample. In further embodiments, the markers are antibodies for PI3K, e.g., p85alpha, p85beta, p110alpha, p110beta, p110gamma, or p110delta subunit. In further embodiments, the markers are fluorescent.

In certain embodiments, the assaying comprises the step of detecting expression of mRNA encoding PI3K e.g., p85alpha, p85beta, p110alpha, p110beta, p110gamma, or p110delta subunit in the sample. In further embodiments, the assaying comprises mixing the sample with a polynucleotide that hybridizes to mRNA encoding PI3K e.g., p85alpha, p85beta, p110alpha, p110beta, p110gamma, or p110delta subunit. In further embodiments, the polynucleotide is conjugated to a fluorescent marker.

In certain embodiments, the assaying comprises moving the sample through separation medium and detecting PI3K e.g., p85alpha, p85beta, p110alpha, p110beta, p110gamma, or p110delta subunit, phosphatidylinositol 3-phosphate, mRNA encoding PI3K e.g., p85alpha, p85beta, p110alpha, p110beta, p110gamma, or p110delta subunit or general PI3K activity. In certain embodiments, the separation medium is an immunosorbent. In certain embodiments, one detects greater than two fold PI3K activity compared to normal.

In certain embodiments, the sample comprises a lymphocyte or fibroblast or does not contain a neuron or brain cell.

### BRIEF DESCRIPTION OF THE FIGURES

FIGS. 1A-E show data suggesting exaggerated PI3K activity and signaling at Fmr1 KO synapses. A, PI3K activity is threefold increased in Fmr1 KO SNS compared with WT SNS, as assessed by the amount of radiolabeled PI3-P on autoradiographies from in vitro PI3K assays (n=8, * p=0.035, paired t test). B, Quantification of phospho-ELK-1 specific Western blots of in vitro ERK1/2 kinase assays from WT and Fmr1 KO SNS using recombinant ELK-1 as a substrate shows no significant change in ERK1/2 activity in the absence of FMRP (n=8, p=0.20, paired t test). FMRP-specific Western blot analyses of the lysate used as starting material for the assay are shown below; tubulin served as loading control. C, Quantitative analysis of recombinant RFP-PH(Akt) domain colocalized with synaptophysin in hippocampal neurons (9-10 DIV) demonstrates increased synaptic localization of RFP-PH(Akt) in Fmr1 KO neurons, suggesting elevated PI3-P3 levels at Fmr1 KO synapses, which lead to a translocation of the fluorescently labeled PH domain (scale bar, 5 μm) (n=5 independent experiments, 6-12 cells each, ***p=0.00002, paired t test). In contrast, total dendritic RFP-PH(Akt) levels were not different between WT and Fmr1 KO neurons. D, E, Phosphorylation of the PI3K downstream signaling molecule Akt (D), but not ERK1/2 (E), is significantly increased in Fmr1 KO SNS compared with WT. Phosphorylation levels were assessed by densitometric analysis of Western blots, and phospho-specific signals were normalized to total levels of the respective proteins (D, n=6, * p=0.014; E, n=7, p=0.18; paired t tests). Representative Western blots are shown below. F-I, PI3K activity and signaling is increased in FMRP-deficient HEK293T cells. In contrast to cortical SNS, HEK293T cells do not express detectable levels of mGluR1 and mGluR5 receptors. F, G, siRNA-mediated knockdown of Fmr1 in HEK293T cells increases PI3K activity significantly (F, n=7, * p=0.039, paired t test), whereas in vitro ERK1/2 assays showed no significant change in activity (G, n=7, p=0.32, paired t test). H, I, Quantification of Akt and ERK1/2 phosphorylation after Fmr1 knockdown demonstrate significantly enhanced phospho-Akt levels (H, n=5, * p=0.018, paired t test) but not phospho-ERK1/2 levels (I, n=6, p=0.29, paired t test). Genotype or siRNA-mediated knockdown was confirmed by Western blotting with an FMRP-specific antibody and a tubulin antibody as loading control (shown below for each experiment). Error bars represent SEM. a.u., Arbitrary unit.

FIGS. 2A-D show data suggesting excess PI3K activity in Fmr1 KO cortical neurons can be reduced by inhibition of gp1 mGluR-mediated signaling. A, Treatment with the mGluR5 antagonist MPEP significantly reduces PI3K activity in Fmr1 KO SNS but not in WT SNS (n=4, two-way ANOVA: genotype, p=0.953; treatment, p=0.425; interaction of genotype and treatment, *p=0.006; LSD post hoc analyses: pwtctr-wtMPEP=0.327, * pwtctr-koctr=0.033, * pkoctr-koMPEP=0.012). B, Disruption of the mGluR5-Homer complex with a tat-fused mGluR5C-terminal peptide but not with a mutated peptide (CT and MUT, respectively; 5 μM) decreases PI3K activity in both WT and Fmr1 KO SNS. Peptide treatment led to a significant decrease in PI3K activity in both WT and KO, but PI3K activity was still significantly increased in KO compared with WT after treatment with the mGluR5C-terminal peptide (n=4, two-way-ANOVA; effect of peptide, *p<0.001; effect of genotype, *p=0.014; interaction of peptide and genotype, p=0.143). Moreover, there was neither a significant genotype-specific difference in PI3K activity in KO samples compared with WT after disruption of the mGluR complex, nor a significant difference in the ratios of KO versus WT PI3K activity when treated with mutated compared to C-terminal peptides. C, PI3K activity in cultured cortical Fmr1 KO neurons is increased compared with WT, and short-hairpin-mediated knockdown of the PI3K enhancer PIKE-L (shPIKE-L) significantly reduces PI3K activity in Fmr1 KO (n=3; two-way ANOVA: genotype, *p=0.001; treatment, *p=0.008; interaction of genotype and treatment, *p=0.014; Tukey's HSD post hoc analyses: *pwtctr-koctr=0.002, * pkoctr-koPIKE=0.007). shPIKE-L did not lead to a complete rescue of PI3K activity to WT levels (KO, 139±14% of WT after PIKE knockdown). Western blots below show knockdown of PIKE-L in WT and KO neurons; tubulin was used as loading control. D, E, Activation of gp1 mGluRs with DHPG (10 min, 100 μM) leads to a twofold increase in PI3K activity in WT SNS (D, n=4, * p=0.033, paired t test) but to decreased PI3K activity in Fmr1 KO SNS (E, n=4, * p=0.049, paired t test).

FIGS. 3A-L show data suggesting p110beta protein expression and translation is regulated by FMRP. A, B, Immunocytochemical analysis of p110beta expression at synapses in WT (A) and Fmr1 KO (B) neurons shows that p110beta (red) colocalizes with the synaptic marker synaptophysin (green), as indicated by yellow signal. Colocalized signal is shown in white (bottom). Scale bar, 20 μm. C, Quantification of p110beta signal intensities overlapping with synaptophysin reveals a significant increase of overlap in Fmr1 KO neurons compared with WT neurons (WT, 27% overlap; KO, 34.9% overlap; n=43 dendrites each for WT and KO, 3 independent hippocampal cultures, **p=0.008, independent t test). Representative images of dendrites from WT and Fmr1 KO that were analyzed for colocalization are shown on the left, with colocalized signal superimposed in white. Scale bar, 3 μm. D, Total levels of synaptophysin were unchanged in Fmr1 KO compared with WT dendrites (n=43, p=0.73, independent t test). E, Quantification of fluorescent p110beta-specific signal within three-dimensional reconstructed synaptophysin punctae reveals increased mean intensity of p110beta-specific signal in synaptophysin punctae in Fmr1 KO dendrites, suggesting specific enrichment of p110beta at single synapses (n=43, ** p=0.004, independent t test). In contrast, the relative number of p110beta-positive synapses was not changed in Fmr1 KO neurons. Example of three-dimensional reconstruction of synaptophysin punctae is shown on the left, and p110beta signal within these punctae is indicated by arrows. Scale bar, 3 μm. F, Densitometric analysis of p110beta-specific Western blots demonstrates increased p110beta protein levels in SNS from Fmr1 KO cortices compared with WT. Signal intensities were normalized to tubulin (n=5, * p=0.043, paired t test); a representative western blot is shown at the right. G, siRNA-mediated reduction of FMRP expression in HEK293T cells leads to increased p110beta protein levels. Signal intensities were normalized to tubulin (n=5, * p=0.005, paired t test). H, FMRP-specific quantitative coimmunoprecipitation from WT and Fmr1 KO brain lysates demonstrates a specific enrichment of p110beta mRNA in WT immunoprecipitations, whereas NR1 mRNA is not enriched. mRNA levels were quantified by quantitative real-time PCR (n=6, two-way ANOVA: pmRNA=0.488, pgenotype=0.372, * pbetween subjects=0.034; Tukey's HSD post hoc analyses: *pp110beta=0.003, pNR1=0.884). I, Recombinant p110beta 3′ UTR fused to EGFP expressed in HEK293T cells is significantly enriched in anti-flag pulldowns with coexpressed flag-tagged mCherry-FMRP but not with flag-tagged mCherry. No specific enrichment can be detected with β-actin 3′ UTR. mRNA levels in pulldowns were quantified by qRT-PCR with EGFP-specific primers and normalized to input (n=4, two-way ANOVA: *pmRNA=0.006, * prec.protein=0.001, * pbetween subjects=0.01; Tukey's HSD post hoc analyses: *pp110beta=0.001, pβ-actin=0.798). J, K, qRT-PCR quantification of mRNA levels in sucrose gradients from SNS shows that p110beta mRNA in polysomal fractions is puromycin sensitive (J, n=3, * p=0.033, paired t test) and significantly increased in these fractions from Fmr1 KO SNS compared with WT; PSD95 and NR1 mRNAs served as positive and negative controls, respectively (K, n=5; PSD95, * p=0.029; NR1, p=0.89; p110beta, *p=0.043, paired t tests; results were normalized to WT). L, Likewise p110beta mRNA is shifted into actively translating polysomes after Fmr1 knockdown in HEK293T cells (n=3, two-way ANOVA: *pgenotype=0.002, * ptreatment <0.001, * pbetween subjects=0.002; Tukey's HSD post hoc analyses: *pctruntr-ctrpuro=0.002, * pctruntr-KDuntr=0.001, * pKDuntr-KDpuro <0.001). All error bars represent SEM.

FIGS. 4A-D show data suggesting dysregulated gp1 mGluR-dependent p110beta expression in Fmr1 KO. A, B, DHPG treatment (15 min, 50 μM) increases p110beta protein levels in WT SNS (A, normalized to control; n=6, * p=0.008, paired t test) and leads to a shift of p110beta mRNA into puromycin-sensitive fractions (B, percentage of total mRNA in polysomal fractions; n=6, * p=0.028, paired t test). C, D, In Fmr1 KO SNS, DHPG treatment does not increase protein expression (C, normalized to control, n=5, p=0.613, paired t test) or enhance association of p110beta mRNA with puromycin-sensitive polysomes (D, percentage of total mRNA in polysomal fractions, n=3, p=0.21, paired t test).

FIGS. 5A-L show data suggesting PI3K antagonists rescue dysregulated basal and stimulus-induced synaptic translation rates in Fmr1 KO SNS. A, Basal translation in cortical Fmr1 KO SNS is increased 30% compared with WT. Translation rates were analyzed by metabolic labeling of SNS with [35S]methionine, and radioactivity incorporation during a 5 min time period was quantified (n=6, * p=0.03, paired t test). B, C, Pretreatment of SNS with an mGluR5 antagonist (B: MPEP, 10 μM) or an mGluR1 antagonist (C: LY367385, 10 μM) significantly decreased translation rates in Fmr1 KO but not in WT SNS (MPEP: n=8, * pwt-ko untreated=0.043, * pko untreated-treated=0.002; LY367385: n=5, * pwt-ko untreated=0.04, * pko untreated-treated=0.007; Tukey's HSD post hoc tests; two-way ANOVAs detect significant interaction between genotype and treatment; MPEP: *p<0.001; LY367385: * p<0.001). D, Treatment with a specific NMDAR antagonist (APV, 50 μM) affected translation similarly across genotypes (n=3; two-way ANOVA: no significant interaction between genotype and treatment; p=0.782). E, F, Treatment of SNS with two different PI3K antagonists, LY294002 (E; 50 μM) and wortmannin (F; 100 nM), significantly reduced amino acid incorporation rates in Fmr1 KO but not in WT (n=6; E, LY294002: * pwt-ko untreated=0.001, * pko treated-untreated=0.026; F, wortmannin: *pwt-ko untreated <0.001, * pko treated-untreated=0.002; Tukey's HSD post hoc tests; two-way ANOVA: significant interaction between genotype and treatment; LY294002: * p<0.001; wortmannin: *p<0.001). LY303511, an inactive analog of LY294002, did not alter translation rates. G, An ERK1/2 antagonist (U0126, 20 μM) did not show a genotype-specific effect on translation rates [n=5, two-way ANOVA: significant effects of treatment (*p=0.001) and genotype (*p=0.001) but no significant interaction between genotype and treatment (p=0.68)]. H, DHPG-induced translational activation in Fmr1 KO SNS is occluded and can be rescued by antagonizing PI3K signaling (wortmannin) but not by ERK1/2 inhibition (U0126) (wortmannin: n=4, one-way ANOVA, *p=0.012, Tukey's HSD post hoc tests: *puntrctr-wortctr=0.045, pwortctr-wortDHPG=0.010; U0126: n=4, one-way ANOVA, *p=0.001, Tukey's HSD post hoc tests: *puntrctr-untrDHPG=0.029, * puntrctr-U0126ctr/DHPG=0.006/0.001, * puntrDHPG-U0126ctr/DHPG=0.019/0.004). I, Inhibitors of PI3K signaling (Wort) and ERK1/2 signaling (U0126) abolish DHPG-induced (15 min, 100 μM) translational activation in WT SNS (wortmannin: n=4, one-way ANOVA, *p=0.002, Tukey's HSD post hoc tests: *puntrctr-untrDHPG=0.006, * pwortctr-wortDHPG=0.004; U0126: n=3, one-way ANOVA, *p<0.001, Tukey's HSD post hoc tests: *puntrctr-untrDHPG=0.029, * puntrctr/DHPG-U0126DHPG=0.022/0.001). Results were normalized to control. Error bars represent SEM.

FIGS. 6A-E show data suggesting PI3K antagonist rescues increased GluR1 endocytosis in Fmr1 KO neurons. A, Representative images illustrate surface and internalized GluR1 staining in WT and Fmr1 KO primary hippocampal neurons under control conditions and after treatment with the PI3K inhibitor LY294002. Scale bar, 50 μm. B, Box-and-whisker plot of the distribution of constitutive endocytosis of AMPARs in distal dendrites shows that enhanced constitutive endocytosis of AMPARs in Fmr1 KO neurons is corrected by application with the PI3K inhibitor LY294002 (50 μM for 1 h; median: WT, 35.7; KO, 44.5; WT+LY294002, 35.3; KO+LY294002, 35.6; n=30 each; one-way ANOVA with Bonferroni's post hoc tests: *p<0.0001, ** p<0.00001). C, Scatter plot of correlations between FMRP signals and endocytosis of AMPARs in distal dendrites shows that AMPAR internalization in Fmr1 KO is reduced by LY294002 (see red symbols, compressed on left side). Scatter plot of signals in each distal dendrite of WT neurons show substantial variation of FMRP signals. Note however that the enhanced endocytosis of AMPARs, detected if FMRP signals are relatively low, is also corrected with LY294002 application. D, Box-and-whisker plot of the distribution of constitutive endocytosis of AMPARs in distal dendrites show that enhanced constitutive endocytosis of AMPARs in Fmr1 KO neurons is not affected by application of the ERK1/2 inhibitor U0126 (20 μM for 1 h; median: WT, 40.2; KO, 50.5; WT+U0126, 43.8; KO+U0126, 53.0; n=30 each; one-way ANOVA with Bonferroni's post hoc test: ***p<0.0001). E, Scatter plot of correlations between FMRP signals and endocytosis of AMPARs in distal dendrites show substantial variation of FMRP signals in WT. Note that the enhanced endocytosis of AMPARs, detected if FMRP signals are relatively low, is not affected with U0126 application.

FIGS. 7A-C show data suggesting a PI3K antagonist rescues increased dendritic spine density in Fmr1 KO neurons. A, Three-dimensional reconstruction of representative dendrites from WT and Fmr1 KO neurons (18 DIV) after 3 d of treatment with vehicle (ctr) or LY294002 (LY, 10 μM) illustrates that increased protrusion density in Fmr1 KO is rescued by PI3K inhibition. Scale bar, 5 μm. B, Automated quantification using FilamentTracer software (Imaris, Bitplane) shows significantly increased protrusion density in vehicle-treated Fmr1 KO, which can be restored to WT levels by LY294002 treatment, but does not change spine morphology in WT. Bar diagrams represent average spine number per 100 μm (WT control, 38.2; WT LY294002, 38.7; KO control, 46.0; KO LY294002, 33.6; n=30, 2 independent cultures, two-way ANOVA: significant interaction between genotype and treatment, *p=0.001; LSD post hoc tests: *pwtctr-koctr=0.024, *** pkoctr-koLY294002<0.0001, pwtctr-wtLY294002=0.996; error bars represent SEM). C, Examples of a dendrite that was analyzed with FilamentTracer (Imaris; Bitplane) illustrate accuracy of the applied method to identify protrusions. Top, Three-dimensional reconstruction of fluorescent signals from GFP-Lifeact transfected hippocampal dendrite; middle, traced and rebuilt dendrite (white) with spines (blue); bottom, overlay of rebuilt dendrite with original image. Scale bar, 10 μm.

FIGS. 8A-C illustrate a proposed model for dysregulated mGluR signaling in FXS. A, B Regulation of the gp1 mGluR-dependent signal pathways PLC/ERK and PI3K/mTOR in WT. A, Under basal conditions, FMRP puts the break on PI3K activity. This is partially attributable to FMRP-mediated repression of p110beta mRNA translation and synaptic localization of the catalytic subunit p110beta (1). Additional mechanisms might include regulation of p110beta-modulating subunits such as p8513 (2) and PIKE (3) by FMRP. B, gp1 mGluR-mediated activation of PLC and PI3K pathways at the synapse. PLC and PI3K share the same substrate PIP2 in the membrane to produce either IP3 and DAG, or PIP3, respectively. The PLC product DAG can activate PKC, leading to induction of the mitogen-activate protein kinase kinase (MEK)/ERK pathway, whereas the PI3K product PIP3 recruits PH-containing kinases PDK1 and Akt to the membrane, thereby inducing their phosphorylation followed by activation of downstream signaling molecules including mTOR. Both pathways induce protein synthesis. During gp1 mGluR stimulation, PLC is activated by small G-proteins. PI3K was shown to be activated by at least two different mechanisms, the Homer-PIKE complex (4) and small G-proteins (5). Furthermore, gp1 mGluR stimulation leads to transient removal of FMRP-mediated translational inhibition by dephosphorylation of FMRP. It is believed that during this time window, synapses experience a twofold “boost” of PI3K activity composed of newly synthesized catalytic p110beta subunits (1) as well as activation of preexisting and newly synthesized PI3K subunit molecules via PIKE and G-proteins (4 and 5). Together, enhanced ERK and PI3K activity lead to increased synaptic protein synthesis. C, The “molecular brake” FMRP is absent in Fmr1 KO, and FMRP-mediated inhibition of p110beta translation and PI3K activity is removed constitutively. Increased p110beta protein levels at synapses (1), which can be activated by basal levels of mGluR signaling (4 and 5), contribute to exaggerated PI3K signaling. Additionally, PI3K activity could be increased by dysregulation of p110beta-modulating subunits (2 and 3), especially PIKE, in the absence of FMRP. DHPG-mediated transient increase in p110beta protein expression is abolished and may partially account for loss of gp1 mGluR-dependent activation of PI3K signaling. Loss of this combined “brake” on PI3K signaling in FXS would elevate PI3K-dependent protein synthesis to a saturated level, which cannot be further increased by gp1 mGluR stimulation.

FIGS. 9A-B shows data suggesting exaggerated PI3K enzymatic function is independently of gp1 mGluRs. PI3K activity and signaling is increased in FMRP-deficient HEK293T cells. In contrast to cortical SNS, HEK293T cells do not express detectable levels of mGluR1 and mGluR5 receptors (9A). siRNA mediated knockdown (1(D) of Fmr 1 in HEK293T cells increases PI3K activity significantly (9B, n=7, * p=0.039; paired t-test).

### DETAILED DESCRIPTION

Studies disclosed herein indicate that excess PI3K activity represents a key cause of the dysfunction in cellular signaling in FXS Inhibition of PI3K can correct defects in cellular signaling that are responsible for disease phenotypes in a mouse model of FXS. Since other forms of autism and autism spectrum disorders (ASDs) also involve genetic, epigenetic or environmental perturbation of PI3K subunits genes and encoded proteins, these findings that treatment of PI3K dysregulation may be effective in the treatment of other ASDs related to FXS.

It has been discovered that FMRP controls the mRNA translation, synaptic localization, and enzymatic activity of the catalytic PI3K subunit p110beta. This provides an initial indication toward a molecular mechanism underlying exaggerated signaling and dysregulated protein synthesis in FXS. In the absence of FMRP, PI3K is overactive at synapses, leading to excessive protein synthesis and occlusion of agonist-induced protein synthesis. FMRP restrains the common downstream signaling molecule PI3K, which may explain features of dysregulated translation mediated by other neurotransmitters. Antagonists of PI3K were shown to correct Fragile X phenotypes in a mouse model that are relevant to human patients with FXS, i.e., dendritic spine defects, and are thus indicated as a viable therapeutic strategy for treating FXS and related neurological disorders such as autism and autism spectrum disorders ASDs.

Excessive PI3K Activity and Increased Synaptic p110Beta Levels in the Absence of FMRP May Underlie Exaggerated mGlur-Mediated Protein Synthesis

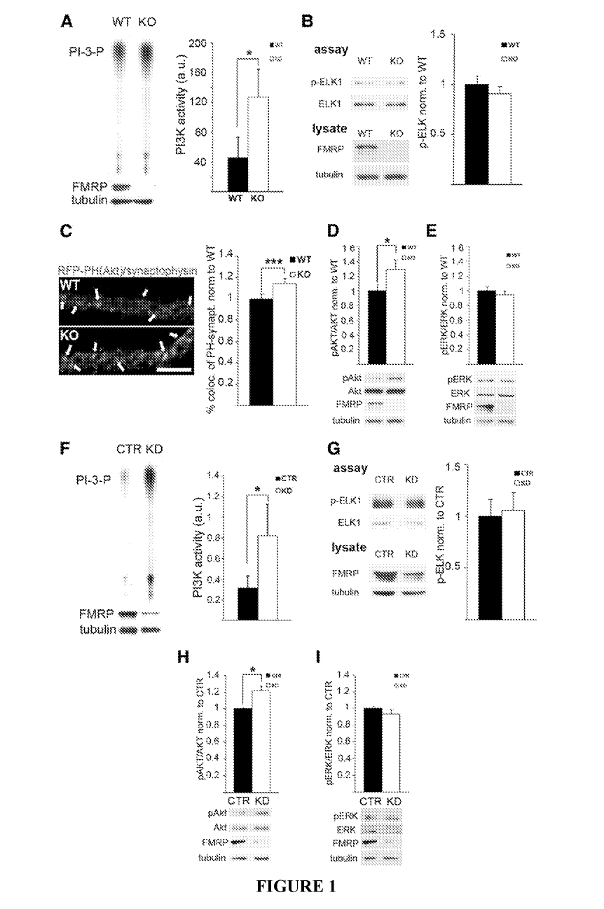

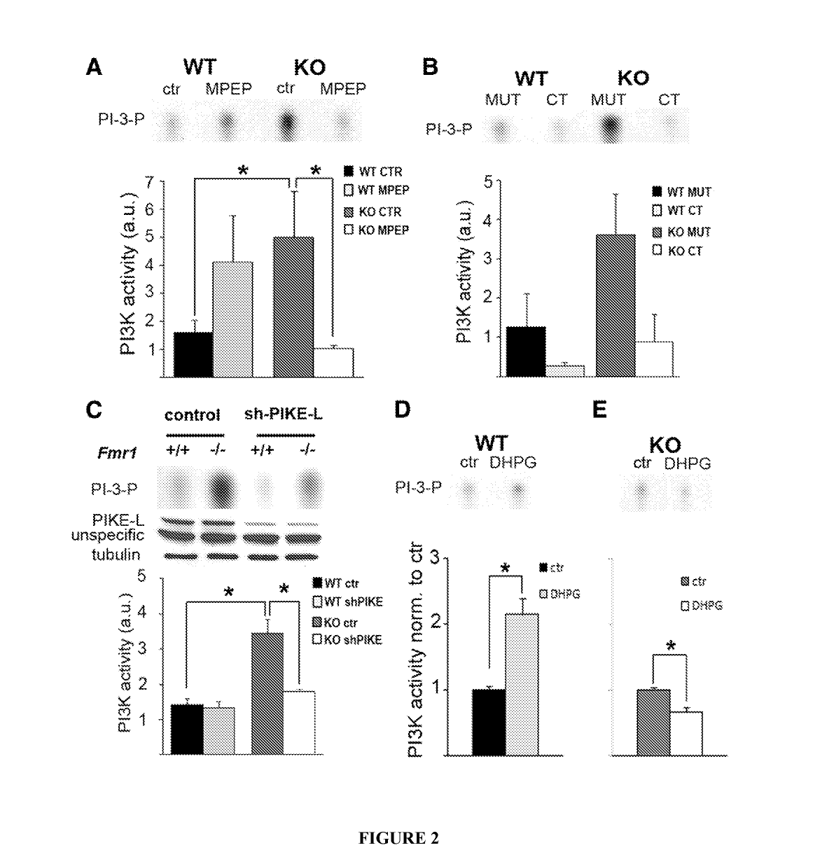

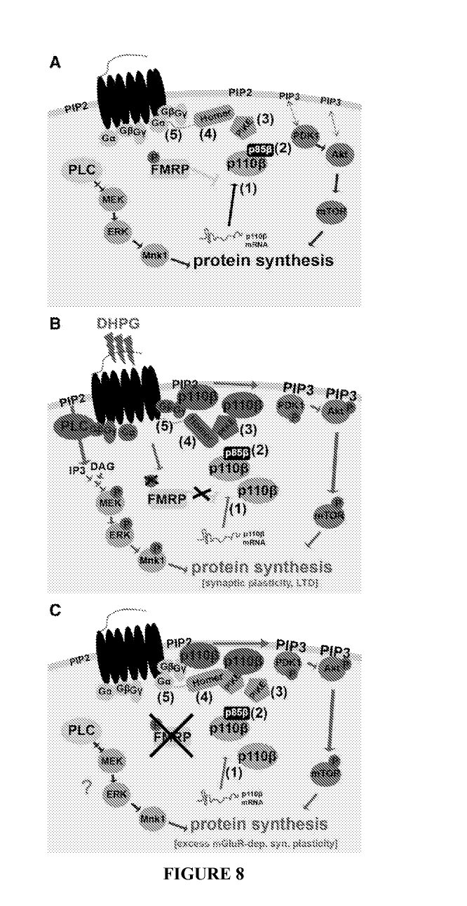

The mGluR theory of FXS proposes that many FXS-related phenotypes in mice and, by extension, human patients originate in exaggerated signaling through gp1 mGluRs. However, the underlying molecular mechanisms have not been revealed so far. Here, data shows that FMRP deficiency results in excessive basal PI3K activity at synapses (FIG. 1A,C), which may represent the cause of the exaggerated mGluR-mediated protein synthesis in FXS. It is hypothesize that elevated synaptic p110beta protein levels in the absence of FMRP increase the amount of p110beta subunits recruited into gp1 mGluR signaling complexes at synapses under basal conditions and thus contribute to excess PI3K activity resulting in exaggerated mGluR signaling and increased basal protein synthesis in FXS (FIG. 8). This hypothesis is supported by data showing that excessive PI3K activity in the absence of FMRP is modulated but not driven or caused by overactive gp1 mGluRs. Excess PI3K activity in the absence of FMRP can occur independently of gp1 mGluRs (FIG. 1F). Furthermore, the results demonstrate that perturbation of mGluR-Homer-PIKE signaling decreases PI3K activity in both WT and KO but cannot reduce the observed exaggerated PI3K activity in Fmr1 KO to WT levels (FIG. 2B). It is believed that during blockage of the mGluR5-Homer-PIKE pathway in Fmr1 KO, excess p110beta protein can still be activated via small G-proteins, a known alternative pathway mediating gp1 mGluR-induced activation of PI3K (FIG. 8). This alternative pathway may be sufficient to induce LTD in Fmr1 KO during blockage of Homer signaling because of excess PI3K. However, mGluR-Homer signaling is important for LTD in WT because PI3K activity is limited by FMRP.

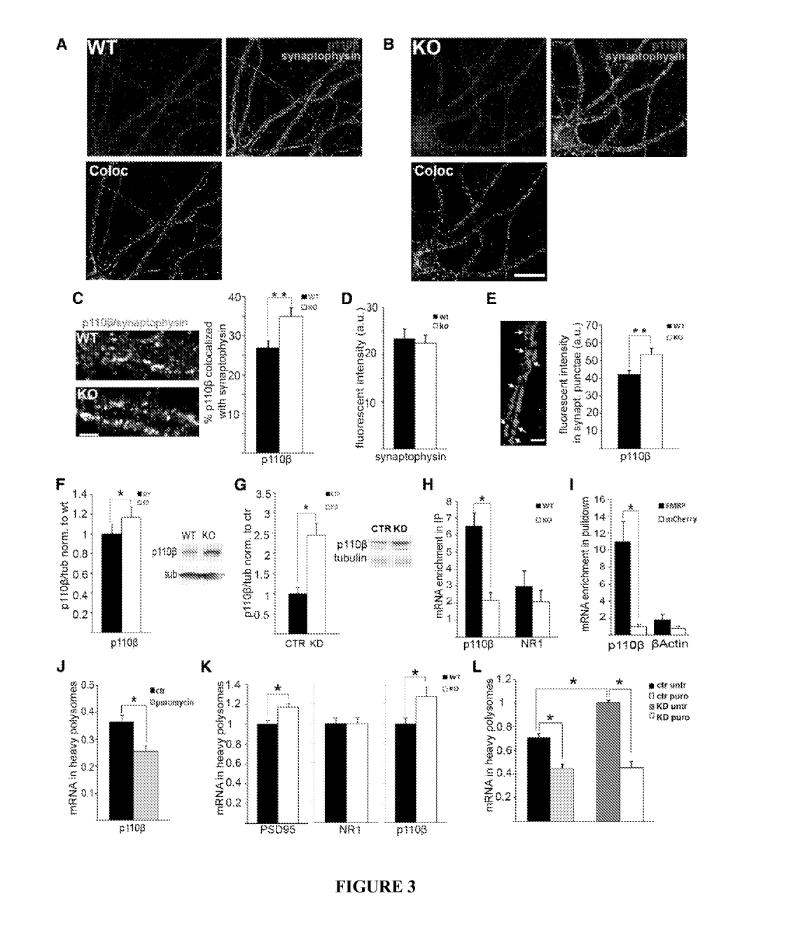

Analysis of synaptic p110beta localization in cultured Fmr1 KO neurons demonstrates that p110beta intensity within synaptophysin punctae is increased in Fmr1 KO, whereas the relative number of p110beta-positive synapses was unchanged (FIG. 3E). These data indicate that, in the absence of FMRP, the population of synapses recruiting p110beta into activated receptor complexes is unchanged but that, because of increased p110beta protein levels in dendrites, more p110beta subunits are translocated into these p110beta-recruiting receptor complexes. This suggests that the number of synapses containing actively signaling gp1 mGluRs is not increased in FMRP-deficient neurons and is in line with the hypothesis that excess PI3K activity at FXS synapses is not caused by generally elevated mGluR activity but rather by increased synaptic p110beta expression.

This disclosure provides evidence for a molecular mechanism that could also account for other dysregulated neurotransmitter-mediated signaling pathways in FXS. Aberrant regulation of p110beta provides a molecular explanation for several recent studies in Fmr1 KO mice, which reported dysregulated neuronal plasticity and signal transduction downstream of the mGluR1/5-Homer-PIKE complex, of D1 dopamine receptors, of Gq-proteins, and of Ras. PI3K regulates all of these pathways. Aberrant signal transduction in FXS is caused by exaggerated p110beta protein levels rather than dysregulated upstream receptors leading to PI3K activation. Although upregulated PIKE-L levels should contribute to excess PI3K activity downstream of gp1 mGluRs (FIG. 2C), they do not explain dysregulation of other receptor pathways.

Translational Regulation of p110Beta mRNA as a Novel Mechanism for gp1 mGluR-Activated Signaling in WT and Dysregulated gp1 mGluR Signaling in Fmr1 KO

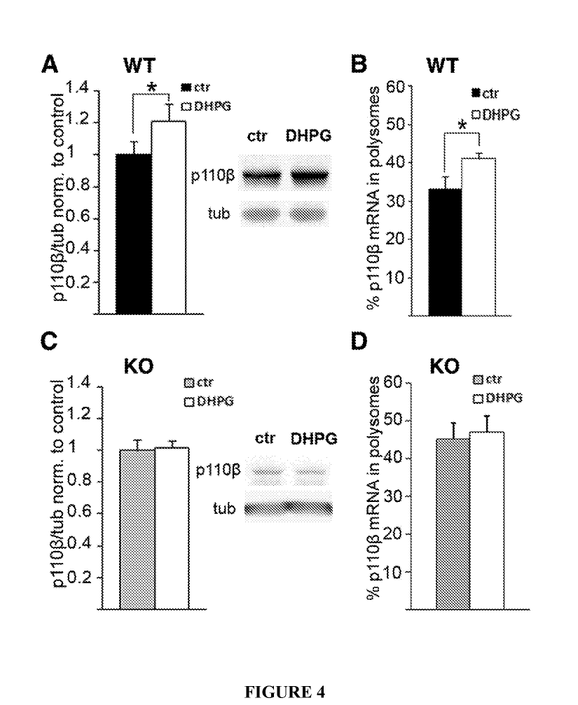

Data suggest that gp 1 mGluR stimulation increases mRNA translation and protein levels of the catalytic subunit p110beta in WT SNS but not in Fmr1 KO SNS (FIG. 4). This reveals a previously unknown mechanism for gp1 mGluR-mediated stimulation of PI3K activity, suggesting that, in WT, gp1 mGluR signaling increases PI3K activity by at least two different mechanisms: (1) recruitment of preexisting p110beta into synaptic receptor protein complexes and (2) increased synthesis of p110beta protein as shown here (FIG. 4A,B). Furthermore, it suggests that increased p110beta mRNA translation contributes to the excess PI3K activity in FXS. These experiments are the first to indicate that the loss of FMRP leads to altered functional activity of a protein encoded by its target mRNA. Apart from the FMRP target mRNA p110beta, an inhibitory subunit of PI3K, p8513, was identified in a screen for putative FMRP targets (Brown et al., (2001) Cell 107:477-487.). Data herein suggests FMRP associates with PIKE-L mRNA and that PIKE-L protein levels are increased at Fmr1 KO synapses. FMRP might thus regulate PI3K activity at multiple steps (FIG. 8).

### PI3K Versus ERK Signaling in FXS

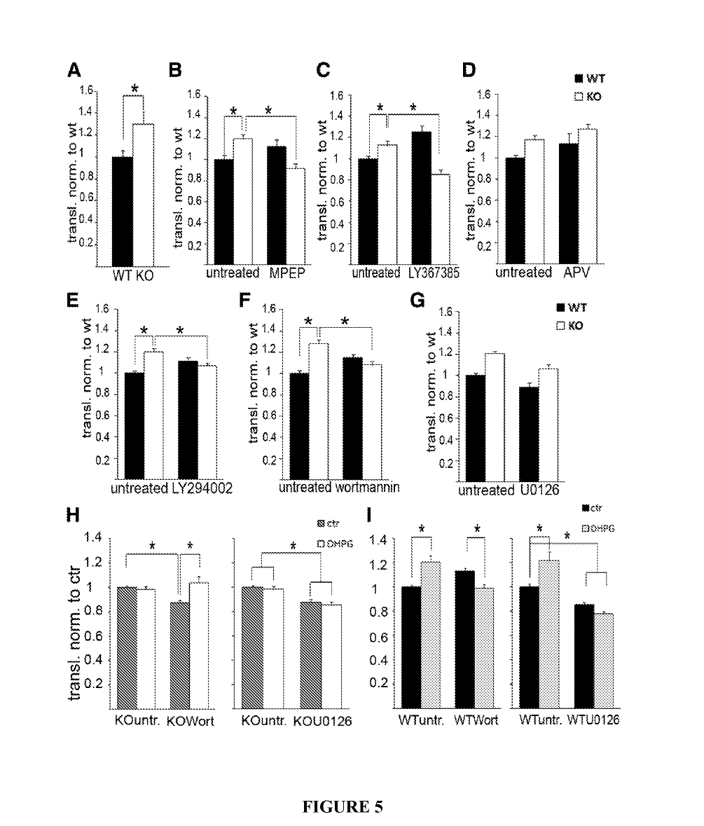

In neurons, the PI3K/mTOR pathway and the PKC/ERK pathway represent two main targets downstream of gp1 mGluRs driving the activation of protein synthesis. This study provides experimental evidence that basal ERK1/2 activity is not altered in SNS from Fmr1 KO mice, whereas PI3K activity is significantly increased. Of note, two studies suggested that loss of FMRP leads to dysregulated ERK1/2 phosphorylation (Hou et al., (2006) Neuron 51:441-454; Kim et al., (2008) Proc Natl Acad Sci USA 105:4429-4434); however, neither study directly examined and compared PI3K and ERK1/2 activity. gp1 mGluR activation of ERK1/2 is mediated by PKC, which in turn is activated by phospholipase C(PLC) (FIG. 8B). PI3K and PLC compete for the same substrate in the membrane, PI(4,5)P2, and excessive PI3K protein at Fmr1 KO synapses could therefore tip the balance between these two pathways toward PI3K signaling. Data disclosed herein support the important role played by ERK1/2 for mGluR-stimulated protein synthesis in WT (FIG. 5G), but indicate that it is not the ERK pathway that is primarily dysregulated in FXS. Interfering with PI3K signaling, but not ERK1/2, rescues dysregulated synaptic translation and corrects exaggerated constitutive internalization of AMPA receptors in Fmr1 KO neurons, supporting the hypothesis that FMRP has a direct effect on PI3K activity but not on ERK1/2.

### PI3K Antagonists as Therapeutics for Diseases Related to Neuronal Defects

An important phenotype in FMRP-deficient neurons is dysregulated synaptic protein synthesis, which results in increased basal protein synthesis in brain slices and at synapses, and loss of neurotransmitter-induced protein synthesis (FIG. 5). Data herein suggest that FMRP controls protein synthesis-dependent regulation of synaptic morphology and function through regulation of PI3K signaling. PI3K antagonists can reduce exaggerated basal translation at Fmr1 KO synapses and restore translational responsiveness to mGluR stimulation. Dysregulated synaptic protein synthesis was proposed to cause many, if not all, neuronal defects in FXS. Reducing PI3K signaling pathways is a promising therapeutic strategy to treat FXS in humans. PI3K antagonists can rescue two protein synthesis-dependent and physiologically relevant phenotypes in hippocampal Fmr1 KO neurons: increased number of internalized AMPA receptors and excessive dendritic spine density. Altered spine morphology is a prominent FXS-associated phenotype, which is not only observed in all animal models for FXS but importantly also in human patients. Importantly, increased spine density in Fmr1 KO can be rescued using doses of LY294002 (10 μM, five times lower than the usual dose) in a 3 d treatment without affecting WT neurons. The absence of any effect on WT neurons after chronic treatment with a PI3K inhibitor over several days corroborates the potential therapeutic value of antagonizing PI3K.

PI3K inhibitors have been studied as anti-cancer and anti-inflammation drugs, including the development of isoform-specific p110beta inhibitors such as 3,3′-(2,4-diaminopteridine-6,7-diyl)diphenol (Crabbe et al. (2007) Trends Biochem Sci 32:450-456). Thus, targeting p110beta directly may be a more precise therapeutic strategy in FXS than the current focus on the upstream gp1 mGluR target.

### Small Molecule PI3K Antagonists

In certain embodiments, the invention relates to the use of PI3K antagonist in the treatment of diseases related neurological defects. Typical PI3K antagonist are described herein, but it is not intended that certain embodiments of the invention be limited to any particular antagonist.

A number of specific PI3K inhibitors/antagonist contemplated for certain embodiments of the invention are those described in U.S. Pat. No. 5,480,906, U.S. Pat. No. 7,723,375, U.S. Pat. No. 7,081,475, U.S. Pat. No. 7,446,124, U.S. Pat. No. 7,767,669, U.S. Pat. No. 7,750,003, U.S. Pat. No. 7,511,041, U.S. Pat. No. 7,696,213, U.S. Pat. No. 7,776,856, U.S. Pat. No. 7,696,204, U.S. Pat. No. 7,781,433, US App Pub Numbers 2008/0249123, US 2010/0210646, US 2010/0130496, US 2010/0016306 US 2009/0258882, US 2010/0227858, US 2009/0286779, US 2009/0018134, US 2010/0150827, US 2010/0227800, US 2009/0325957, US 2009/0306126, US 2008/0039459, US 2008/0132502, US 2010/0249110, US 2010/0249120, US 2010/0249109, US 2010/0249153, US 2010/0249108, US 2010/0249127, US 2010/0249115, US 2008/0076758, US 2008/0039459, US 2008/0242665, US 2008/0269210, US 2010/0172867, US 2010/0239526, US 2010/023952, PCT App Pub Numbers WO/2010/102958, WO/2009/147190, WO/2009/147188, WO/2009/147189, WO/2009/147187, WO/2010/109084, WO/2010/092015, WO/2010/091996, WO/2010/057877, WO/2010/056320, WO/2010/037765, WO/2010/007100, WO/2010/007099, WO/2010/005558, WO/2009/099163, WO/2009/120094, WO/2009/091550, WO/2009/007750, WO/2008/070150, WO/2010/114484, WO/2010/114494, WO/2009/157880, WO/2009/093981, WO/2009/045175, WO/2009/045174, WO/2008/044045, WO/2009/055730, WO/2009/120094, WO/2009/091550, WO/2008/070150, WO/2010/038165, and WO/2007/044729 all hereby incorporated by reference.

### Antibodies to PI3K

In certain embodiments, the invention relates to pharmaceutical compositions with an antibody that specifically binds a PI3K, or a biologically active fragment thereof, such as p110beta for, use in the treatment of neurological disorders.

In certain embodiments, the invention should not be construed as being limited solely one type of antibody. Rather, should be construed to include antibodies, as that term is defined elsewhere herein, that specifically bind to PI3K, p110beta subunit or portions thereof. Further, in certain embodiments, the present invention should be construed to encompass antibodies that are able to bind PI3K, p110beta subunit or portions thereof on Western blots, in immunohistochemical staining of tissues thereby localizing PI3K, p110beta subunit or portions thereof in the tissues, and in immunofluorescence microscopy of a cell transiently transfected with a nucleic acid encoding at least a portion of the polypeptide corresponding to PI3K, p110beta subunit, or portions thereof.

One skilled in the art would appreciate, based upon the disclosure provided herein, that the antibody can specifically bind with any portion of the polypeptide corresponding to the PI3K, p110beta subunit or portions thereof and the polypeptide can be used to generate antibodies specific therefor. However, in certain embodiments, invention is not limited to using the full-length polypeptide corresponding to PI3K, p110beta subunit or portions thereof as an immunogen. In certain embodiments, the antibody binds to an epitope that is a continuous 5 amino acid sequence within SEQ ID NO: 15.

The antibodies can be produced by immunizing an animal such as, but not limited to, a rabbit or a mouse, with a protein, or a portion thereof, or by immunizing an animal using a protein comprising at least a portion of the polypeptide corresponding to PI3K, p110beta subunit or portions thereof such as a continuous 5 amino acid sequence within SEQ ID NO: 15. One skilled in the art would appreciate, based upon the disclosure provided herein that smaller fragments of these proteins can also be used.

The antibody can be used to detect and/or measure the amount of protein present in a biological sample using well-known methods such as, but not limited to, Western blotting and enzyme-linked immunosorbent assay (ELISA). The antibody can also be used to immunoprecipitate and/or immuno-affinity purify their cognate antigen using methods well-known in the art. Thus, by administering the antibody to a cell or to the tissue of an animal, or to the animal itself, the interactions between PI3K, p110beta subunit or portions thereof and its cognate receptor are therefore inhibited.

In another embodiment, a monoclonal antibody is obtained from the non-human animal, and then modified, e.g., humanized, deimmunized, chimeric, antibody may be produced using recombinant DNA techniques known in the art. A variety of approaches for making chimeric antibodies have been described. See, e.g., U.S. Pat. No. 4,816,567 and U.S. Pat. No. 4,816,397. Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

In certain embodiments, a humanized antibody is optimized by the introduction of conservative substitutions, consensus sequence substitutions, germline substitutions and/or backmutations. An antibody or fragment thereof may also be modified by specific deletion of human T cell epitopes or “deimmunization” by the methods disclosed in U.S. Pat. No. 7,125,689 and U.S. Pat. No. 7,264,806. Briefly, the heavy and light chain variable domains of an antibody can be analyzed for peptides that bind to MHC Class II; these peptides represent potential T-cell epitopes. For detection of potential T-cell epitopes, a computer modeling approach termed “peptide threading” can be applied, and in addition a database of human MHC class II binding peptides can be searched for motifs present in the VH and VL sequences. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. The V BASE directory provides a comprehensive directory of human immunoglobulin variable region sequences. These sequences can be used as a source of human sequence, e.g., for framework regions and CDRs. Consensus human framework regions can also be used, e.g., as described in U.S. Pat. No. 6,300,064.

### Nucleic Acid Interference of PI3K

In certain embodiments, the invention relates to compounds, compositions, and methods useful for modulating PI3K or p110beta subunit expression using short interfering nucleic acid (siNA) molecules. Examples are provided in US. Pub App No. 20090258929 and PCT Pub App WO/2009/152346 both hereby incorporated by reference. Particular embodiments of the invention relate to small nucleic acid molecules, such as short interfering nucleic acid (siNA), short interfering RNA (siRNA), double-stranded RNA (dsRNA), micro-RNA (miRNA), and short hairpin RNA (shRNA) molecules and methods used to modulate the expression of PI3K, p110, or p110beta subunit.

RNA interference refers to the process of sequence specific post-transcriptional gene silencing in animals mediated by short interfering RNAs (siRNAs) sometimes referred to as post-transcriptional gene silencing or RNA silencing. The presence of long dsRNAs in cells is thought to stimulate the activity of a ribonuclease III enzyme referred to as Dicer. Dicer is thought to be involved in the processing of the dsRNA into short pieces of dsRNA known as short interfering RNAs (siRNAs). Short interfering RNAs derived from Dicer activity are typically about 21 to about 23 nucleotides in length and comprise about 19 base pair duplexes. Dicer has also been implicated in the excision of 21- and 22-nucleotide small temporal RNAs (stRNAs) from precursor RNA of conserved structure that are implicated in translational control. The RNAi response is thought to feature an endonuclease complex containing a siRNA, commonly referred to as an RNA-induced silencing complex (RISC), which mediates cleavage of single-stranded RNA having sequence homologous to the siRNA. Cleavage of the target RNA takes place in the middle of the region complementary to the guide sequence of the siRNA duplex. In addition, RNA interference is thought to involve small RNA (e.g., micro-RNA or miRNA) mediated gene silencing, presumably though cellular mechanisms that regulate chromatin structure and thereby prevent transcription of target gene sequences. As such, siNA molecules can be used to mediate gene silencing via interaction with RNA transcripts or alternately by interaction with particular gene sequences, wherein such interaction results in gene silencing either at the transcriptional level or post-transcriptional level.

RNAi has been studied in a variety of systems. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates has revealed certain preferences for siRNA length, structure, chemical composition, and sequence that mediate efficient RNAi activity. These studies have shown that 21 nucleotide siRNA duplexes are typical when using two 2-nucleotide 3′-terminal nucleotide overhangs. Substitution of 3′-terminal siRNA nucleotides with deoxy nucleotides was shown to be tolerated. Other studies have indicated that a 5′-phosphate on the target-complementary strand of a siRNA duplex is beneficial for siRNA activity and that ATP is utilized to maintain the 5′-phosphate moiety on the siRNA. siRNA molecules lacking a 5′-phosphate are active when introduced exogenously.

A siNA can be unmodified or chemically-modified. A siNA can be chemically synthesized, expressed from a vector or enzymatically synthesized. Various chemically-modified synthetic short interfering nucleic acid (siNA) molecules are capable of modulating PI3K or p110beta subunit or portions thereof expression or activity in cells by RNA interference (RNAi).

In one embodiment, the invention relates to a double-stranded short interfering nucleic acid (siNA) molecule that down-regulates expression of PI3K or p110beta subunit or portions thereof, wherein said siNA molecule comprises about 15 to about 35 base pairs.

In some embodiments, the invention relates to nucleic acids obtained by endo-ribonuclease prepared siRNA (esiRNA). A representative endo-ribonuclease is naturally isolated or recombinant bacterial RNase III. Upon purification, one uses the enzyme to generate esiRNAs. One can generate double stranded RNA of PI3K or p110beta subunit mRNA by in vitro transcription. See. Yang et al., (2002), Proc. Natl. Acad. Sci. USA 99(15): 9942-9947. One uses the RNase III to digest the transcripts into smaller fragments. One runs the digested RNA molecules on a gel and RNA duplexes of 15-30 nucleotides are isolated.

In some embodiments, the invention relates to methods of treating a subject by administering a pharmaceutical composition with a heterogeneous mixture of siNAs that are homologous to the PI3K or p110beta subunit or portions thereof mRNA sequence or fragment thereof. In certain embodiments, the fragments have greater than 150 or 200 nucleotides.

In certain embodiment, nucleic acids disclosed herein are expressed in a recombinant vector in vivo contained in the pharmaceutical product. Representative recombinant vectors include plasmids, adenoviral vectors, adeno-associated viral vectors, retroviral vectors, and lentiviral vectors.

### Aptamers of PI3K

In some embodiments, the invention relates to PI3K antagonist that are aptamers of PI3K or the p110beta subunit. In certain embodiments, aptamers are contemplated as molecules that interfere with PI3K signaling. Oligonucleotides can be developed to target PI3K or p110beta. SELEX (“Systematic Evolution of Ligands by Exponential Enrichment”) is a combinatorial chemistry technique for producing oligonucleotides of either single-stranded DNA or RNA that specifically bind to a target. Standard details on generating aptamers can be found in U.S. Pat. No. 5,475,096, and U.S. Pat. No. 5,270,163.

The SELEX process provides a class of products which are referred to as nucleic acid ligands or aptamers, each having a unique sequence, and which has the property of binding specifically to a desired target compound or molecule. Each SELEX-identified nucleic acid ligand is a specific ligand of a given target compound or molecule. The SELEX process is based on the fact that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets. The SELEX method applied to the application of high affinity binding involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand enriched mixture of nucleic acids, then reiterating the steps of binding, partitioning, dissociating and amplifying through as many cycles as desired to yield highly specific high affinity nucleic acid ligands to the target molecule.

The basic SELEX method has been modified to achieve a number of specific objectives. For example, U.S. Pat. No. 5,707,796 describes the use of the SELEX process in conjunction with gel electrophoresis to select nucleic acid molecules with specific structural characteristics, such as bent DNA. U.S. Pat. No. 5,763,177 and U.S. Pat. No. 6,011,577 describe a SELEX based method for selecting nucleic acid ligands containing photoreactive groups capable of binding and/or photocrosslinking to and/or photoinactivating a target molecule. U.S. Pat. No. 5,580,737 describes a method for identifying highly specific nucleic acid ligands able to discriminate between closely related molecules, which can be non-peptidic, termed Counter-SELEX. U.S. Pat. No. 5,567,588 describes a SELEX-based method which achieves efficient partitioning between oligonucleotides having high and low affinity for a target molecule.

The SELEX method encompasses the identification of high-affinity nucleic acid ligands containing modified nucleotides conferring improved characteristics on the ligand, such as improved in vivo stability or improved delivery characteristics. Examples include U.S. Pat. No. 5,660,985 and U.S. Pat. No. 5,580,737.

The SELEX method encompasses combining selected oligonucleotides with other selected oligonucleotides and non-oligonucleotide functional units as described in U.S. Pat. No. 5,637,459 and U.S. Pat. No. 5,683,867. These applications allow the combination of the broad array of shapes and other properties, and the efficient amplification and replication properties, of oligonucleotides with the desirable properties of other molecules.

### Synthesis of Nucleic Acid Molecules

Small nucleic acid motifs (“small” refers to nucleic acid motifs no more than 100 nucleotides in length, preferably no more than 80 nucleotides in length, and most preferably no more than 50 nucleotides in length; e.g., individual siNA oligonucleotide sequences or siNA sequences synthesized in tandem) are preferably used for exogenous delivery. The structure of these molecules increases the ability of the nucleic acid to invade targeted regions of protein and/or RNA structure.

One synthesizes oligonucleotides (e.g., certain modified oligonucleotides or portions of oligonucleotides) using protocols known in the art as, for example, described in U.S. Pat. No. 6,001,311. The synthesis of oligonucleotides typically makes use of common nucleic acid protecting and coupling groups, such as dimethoxytrityl at the 5′-end and phosphoramidites at the 3′-end. In a non-limiting example, small scale syntheses are conducted on a 394 Applied Biosystems, Inc. synthesizer using a 0.2 micro mol scale protocol with a 2.5 min coupling step for 2′-O-methylated nucleotides and a 45 second coupling step for 2′-deoxy nucleotides or 2′-deoxy-2′-fluoro nucleotides. Alternatively, syntheses at the 0.2 micro mol scale can be performed on a 96-well plate synthesizer, such as the instrument produced by Protogene (Palo Alto, Calif.) with minimal modification to the cycle. A 33-fold excess of 2′-O-methyl phosphoramidite and a 105-fold excess of S-ethyl tetrazole can be used in each coupling cycle of 2′-O-methyl residues relative to polymer-bound 5′-hydroxyl. A 22-fold excess of deoxy phosphoramidite and a 70-fold excess of S-ethyl tetrazole mop can be used in each coupling cycle of deoxy residues relative to polymer-bound 5′-hydroxyl.

Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM 12, 49 mM pyridine, 9% water in THF (PerSeptive Biosystems, Inc.). S-Ethyltetrazole solution (0.25 M in acetonitrile) is made up from the solid obtained from American International Chemical, Inc. Alternately, for the introduction of phosphorothioate linkages, Beaucage reagent (3H-1,2-benzodithiol-3-one 1,1-dioxide, 0.05 M in acetonitrile) is used.

Deprotection of the DNA-based oligonucleotides is performed as follows: the polymer-bound trityl-on oligonucleotide is transferred to a 4 mL glass screw top vial and suspended in a solution of 40% aqueous methylamine (1 mL) at 65 degrees for 10 minutes. After cooling to -20 degrees, the supernatant is removed from the polymer support. The support is washed three times with 1.0 mL of EtOH:MeCN:H₂O/3:1:1, vortexed and the supernatant is then added to the first supernatant. The combined supernatants, containing the oligonucleotide, are dried.

Alternatively, the nucleic acid molecules can be synthesized separately and joined together post-synthetically, for example, by ligation or by hybridization following synthesis and/or deprotection.

A siNA molecule can also be assembled from two distinct nucleic acid strands or fragments wherein one fragment includes the sense region and the second fragment includes the antisense region of the RNA molecule.

The nucleic acid molecules can be modified extensively to enhance stability by modification with nuclease resistant groups, for example, 2′-amino, 2′-C-allyl, 2′-fluoro, 2′-β-methyl, 2′-H). siNA constructs can be purified by gel electrophoresis using general methods or can be purified by high pressure liquid chromatography and re-suspended in water.

Chemically synthesizing nucleic acid molecules with modifications (base, sugar and/or phosphate) can prevent their degradation by serum ribonucleases, which can increase their potency. See e.g., U.S. Pat. No. 5,652,094, U.S. Pat. No. 5,334,711, and U.S. Pat. No. 6,300,074. All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

In one embodiment, nucleic acid molecules include one or more (e.g., about 1, 2, 3, 4, 5, 6, 7, 8, 9, 10, or more) G-clamp nucleotides. A G-clamp nucleotide is a modified cytosine analog wherein the modifications confer the ability to hydrogen bond both Watson-Crick and Hoogsteen faces of a complementary guanine within a duplex. A single G-clamp analog substitution within an oligonucleotide can result in substantially enhanced helical thermal stability and mismatch discrimination when hybridized to complementary oligonucleotides. The inclusion of such nucleotides in nucleic acid molecules results in both enhanced affinity and specificity to nucleic acid targets, complementary sequences, or template strands. In another embodiment, nucleic acid molecules include one or more (e.g., about 1, 2, 3, 4, 5, 6, 7, 8, 9, 10, or more) LNA “locked nucleic acid” nucleotides such as a 2′,4′-C methylene bicyclo nucleotide (see for example U.S. Pat. No. 6,639,059, U.S. Pat. No. 6,670,461, U.S. Pat. No. 7,053,207).

In another embodiment, the invention features conjugates and/or complexes of siNA molecules. Such conjugates and/or complexes can be used to facilitate delivery of siNA molecules into a biological system, such as a cell. The conjugates and complexes provided may impart therapeutic activity by transferring therapeutic compounds across cellular membranes, altering the pharmacokinetics, and/or modulating the localization of nucleic acid molecules. In general, the transporters described are designed to be used either individually or as part of a multi-component system, with or without degradable linkers. These compounds are expected to improve delivery and/or localization of nucleic acid molecules into a number of cell types originating from different tissues, in the presence or absence of serum (see U.S. Pat. No. 5,854,038). Conjugates of the molecules described herein can be attached to biologically active molecules via linkers that are biodegradable, such as biodegradable nucleic acid linker molecules.

In yet another embodiment, siNA molecules having chemical modifications that maintain or enhance enzymatic activity of proteins involved in RNAi are provided. Such nucleic acids are also generally more resistant to nucleases than unmodified nucleic acids. Thus, in vitro and/or in vivo the activity should not be significantly lowered.

In another aspect a siNA molecule comprises one or more 5′ and/or a 3′-cap structure, for example on only the sense siNA strand, the antisense siNA strand, or both siNA strands. By “cap structure” is meant chemical modifications, which have been incorporated at either terminus of the oligonucleotide. See, for example, Adamic et al., U.S. Pat. No. 5,998,203. These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples, the 5′-cap includes, but is not limited to, glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine and therefore lacks a base at the l′-position.

In one embodiment, the invention features modified siNA molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions.

### Formulations

Pharmaceutical compositions disclosed herein may be in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se (for which reference is made to the references referred to below).

When the compounds of the invention contain an acidic group as well as a basic group, the compounds of the invention may also form internal salts, and such compounds are within the scope of the invention. When a compound contains a hydrogen-donating heteroatom (e.g. NH), salts are contemplated to covers isomers formed by transfer of said hydrogen atom to a basic group or atom within the molecule.

Pharmaceutically acceptable salts of the compounds include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein may be administered in the form of prodrugs. A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds. Methods of structuring a compound as prodrugs can be found in the book of Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids.

Pharmaceutical compositions for use in the present invention typically comprise an effective amount of a compound and a suitable pharmaceutical acceptable carrier. The preparations may be prepared in a manner known per se, which usually involves mixing the at least one compound according to the invention with the one or more pharmaceutically acceptable carriers, and, if desired, in combination with other pharmaceutical active compounds, when necessary under aseptic conditions. Reference is again made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences.

Generally, for pharmaceutical use, the compounds may be formulated as a pharmaceutical preparation comprising at least one compound and at least one pharmaceutically acceptable carrier, diluent or excipient and/or adjuvant, and optionally one or more further pharmaceutically active compounds.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300 or 400 mg per unit dosage.

The compounds can be administered by a variety of routes including the oral, ocular, rectal, transdermal, subcutaneous, intravenous, intramuscular or intranasal routes, depending mainly on the specific preparation used. The compound will generally be administered in an “effective amount”, by which is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which may be administered as a single daily dose, divided over one or more daily doses. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences.

For an oral administration form, the compound can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, the compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant.

For subcutaneous or intravenous administration, the compounds, if desired with the substances customary therefore such as solubilizers, emulsifiers or further auxiliaries are brought into solution, suspension, or emulsion. The compounds of formula I can also be lyophilized and the lyophilizates obtained used, for example, for the production of injection or infusion preparations. Suitable solvents are, for example, water, physiological saline solution or alcohols, e.g. ethanol, propanol, glycerol, sugar solutions such as glucose or mannitol solutions, or mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. When rectally administered in the form of suppositories, the formulations may be prepared by mixing the compounds of formula I with a suitable non-irritating excipient, such as cocoa butter, synthetic glyceride esters or polyethylene glycols, which are solid at ordinary temperatures, but liquefy and/or dissolve in the rectal cavity to release the drug.

In certain embodiments, it is contemplated that these compositions can be extended release formulations. Typical extended release formations utilize an enteric coating. Typically, a barrier is applied to oral medication that controls the location in the digestive system where it is absorbed. Enteric coatings prevent release of medication before it reaches the small intestine. Enteric coatings may contain polymers of polysaccharides, such as maltodextrin, xanthan, scleroglucan dextran, starch, alginates, pullulan, hyaloronic acid, chitin, chitosan and the like; other natural polymers, such as proteins (albumin, gelatin etc.), poly-L-lysine; sodium poly(acrylic acid); poly(hydroxyalkylmethacrylates) (for example poly(hydroxyethylmethacrylate)); carboxypolymethylene (for example Carbopol™); carbomer; polyvinylpyrrolidone; gums, such as guar gum, gum arabic, gum karaya, gum ghatti, locust bean gum, tamarind gum, gellan gum, gum tragacanth, agar, pectin, gluten and the like; poly(vinyl alcohol); ethylene vinyl alcohol; polyethylene glycol (PEG); and cellulose ethers, such as hydroxymethylcellulose (HMC), hydroxyethylcellulose (HEC), hydroxypropylcellulose (HPC), methylcellulose (MC), ethylcellulose (EC), carboxyethylcellulose (CEC), ethylhydroxyethylcellulose (EHEC), carboxymethylhydroxyethylcellulose (CMHEC), hydroxypropylmethyl-cellulose (HPMC), hydroxypropylethylcellulose (HPEC) and sodium carboxymethylcellulose (Na CMC); as well as copolymers and/or (simple) mixtures of any of the above polymers. Certain of the above-mentioned polymers may further be crosslinked by way of standard techniques.

The choice of polymer will be determined by the nature of the active ingredient/drug that is employed in the composition of the invention as well as the desired rate of release. In particular, it will be appreciated by the skilled person, for example in the case of HPMC, that a higher molecular weight will, in general, provide a slower rate of release of drug from the composition. Furthermore, in the case of HPMC, different degrees of substitution of methoxyl groups and hydroxypropoxyl groups will give rise to changes in the rate of release of drug from the composition. In this respect, and as stated above, it may be desirable to provide compositions of the invention in the form of coatings in which the polymer carrier is provided by way of a blend of two or more polymers of, for example, different molecular weights in order to produce a particular required or desired release profile.

Microspheres of polylactide, polyglycolide, and their copolymers poly(lactide-co-glycolide) may be used to form sustained-release protein delivery systems. Proteins, such as antibodies or fragments thereof can be entrapped in the poly(lactide-co-glycolide) microsphere depot by a number of methods, including formation of a water-in-oil emulsion with water-borne protein and organic solvent-borne polymer (emulsion method), formation of a solid-in-oil suspension with solid protein dispersed in a solvent-based polymer solution (suspension method), or by dissolving the protein in a solvent-based polymer solution (dissolution method). One can attach poly(ethylene glycol) to proteins (PEGylation) to increase the in vivo half-life of circulating therapeutic proteins and decrease the chance of an immune response.

A siNA or aptamer molecule may be within or on the exterior of a delivery vehicle, including liposomes, for administration to a subject, carriers and diluents and their salts, and/or can be present in pharmaceutically acceptable formulations. U.S. Pat. No. 6,395,713 and U.S. Pat. No. 5,616,490 further describe general methods for delivery of nucleic acid molecules.

Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example U.S. Pat. No. 7,141,540 and U.S. Pat. No. 7,060,498), poly(lactic-co-glycolic)acid (PLGA) and PLCA microspheres (see for example U.S. Pat. No. 6,447,796), biodegradable nanocapsules, and bioadhesive microspheres, or by proteinaceous vectors (U.S. Pat. No. 7,067,632). In another embodiment, the nucleic acid molecules can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

In one embodiment, a siNA or aptamer molecule is complexed with membrane disruptive agents such as those described in U.S. Pat. No. 6,835,393. In another embodiment, the membrane disruptive agent or agents and the siNA molecule are also complexed with a cationic lipid or helper lipid molecule, such as those lipids described in U.S. Pat. No. 6,235,310.

Embodiments of the invention feature a pharmaceutical composition comprising one or more nucleic acid(s) in an acceptable carrier, such as a stabilizer, buffer, and the like. The oligonucleotides can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions can also be formulated and used as tablets, capsules or elixirs for oral administration, suppositories for rectal administration, sterile solutions, suspensions for administration by injection, and the other compositions known in the art.

Embodiments of the invention also feature the use of the composition comprising surface-modified liposomes containing poly (ethylene glycol) lipids (PEG-modified, or long-circulating liposomes or stealth liposomes). These formulations offer a method for increasing the circulation and accumulation of in target tissues. The long-circulating liposomes enhance the pharmacokinetics and pharmacodynamics of DNA and RNA. See U.S. Pat. No. 5,820,873 and U.S. Pat. No. 5,753,613. Long-circulating liposomes are also likely to protect from nuclease degradation.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Alternatively, certain siNA or aptamer molecules can be expressed within cells from eukaryotic promoters. Those skilled in the art realize that any nucleic acid can be expressed in eukaryotic cells from the appropriate DNA/RNA vector. The activity of such nucleic acids can be augmented by their release from the primary transcript by an enzymatic nucleic acid. See U.S. Pat. No. 5,795,778, and U.S. Pat. No. 5,837,542.

In certain embodiments, the invention relates to oligonucleotides expressed from transcription units inserted into DNA or RNA vectors. The recombinant vectors can be DNA plasmids or viral vectors. siNA expressing viral vectors can be constructed based on, but not limited to, lentivirus, adeno-associated virus, retrovirus, adenovirus, or alphavirus. In another embodiment, pol III based constructs are used to express nucleic acid molecules (see for example U.S. Pat. No. 5,902,880 and U.S. Pat. No. 6,146,886). The recombinant vectors capable of expressing the siNA molecules can be delivered as described above, and persist in target cells. Alternatively, viral vectors can be used that provide for transient expression of nucleic acid molecules. Such vectors can be repeatedly administered as necessary. Once expressed, the siNA molecule interacts with the target mRNA and generates an RNAi response. Delivery of siNA molecule expressing vectors can be systemic, such as by intravenous or intra-muscular administration, by administration to target cells ex-planted from a subject followed by reintroduction into the subject, or by any other means that would allow for introduction into the desired target cell.

In certain embodiments, the invention relates to an expression vector comprising a nucleic acid sequence encoding at least one oligonucleotide of the instant invention. The expression vector can encode one or both strands of a siNA duplex, or a single self-complementary strand that self hybridizes into an siNA duplex. The nucleic acid sequences encoding the siNA molecules of the instant invention can be operably linked in a manner that allows expression of the siNA molecule.

In certain embodiments, the invention relates to an expression vector comprising: a) a transcription initiation region (e.g., eukaryotic pol I, II or III initiation region); b) a transcription termination region (e.g., eukaryotic pol I, II or III termination region); and c) a nucleic acid sequence encoding at least one of the siNA molecules of the instant invention, wherein said sequence is operably linked to said initiation region and said termination region in a manner that allows expression and/or delivery of the siNA molecule. The vector can optionally include an open reading frame (ORF) for a protein operably linked on the 5′ side or the 3′-side of the sequence encoding the siNA; and/or an intron (intervening sequences).

Transcription of the siNA molecule sequences can be driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells. More specifically, transcription units such as the ones derived from genes encoding U6 small nuclear (snRNA), transfer RNA (tRNA) and adenovirus VA RNA are useful in generating high concentrations of desired RNA molecules such as siNA in cells. See U.S. Pat. No. 5,624,803 and U.S. Pat. No. 5,672,501. The above siNA transcription units can be incorporated into a variety of vectors for introduction into mammalian cells, including but not restricted to, plasmid DNA vectors, viral DNA vectors (such as adenovirus or adeno-associated virus vectors), or viral RNA vectors.

### Excess PI3K Activity as a Biomarker in FXS and ASDs

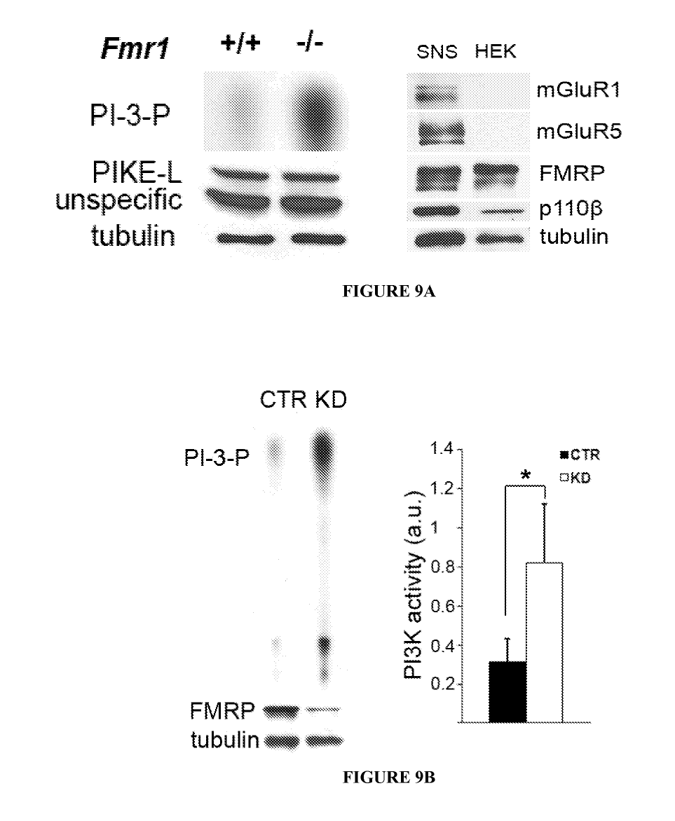

A common feature of FXS-associated neuroanatomical, electrophysiological, behavioral, and biochemical phenotypes is the low magnitude of the observed effects in the mouse model. FXS-associated abnormalities are usually rather subtle, mostly only showing 20-30% difference to wild type animals, which makes their reliable and reproducible detection often difficult and subject to variability. Experimental results show that in Fmr1 KO mouse brain synaptic fractions, in cultured cortical neurons from Fmr1 KO embryos, as well as in human embryonic kidney cells (HEK293T cells) with siRNA-mediated reduced FMRP-expression (FIG. 9), enzymatic activity of the catalytic PI3K subunit p110beta is 2.5-3-fold increased compared to wild type. The HEK293T cells do not express gp1 mGluRs, indicating that excess PI3K activity caused by lack or reduction of FMRP expression does not depend on the presence of gp1 mGluRs, but can be detected in any type of cell.

PI3K activity can be detected in accessible cells such as lymphocytes or fibroblasts. In one embodiment of the invention, one employs an assay involving immunoprecipitation of p110beta, incubation with phospho-inositol and radiolabeled ATP, followed by thin-layer chromatography and autoradiographic detection of the product phosphatidylinositol 3-phosphate (PIP3). Other methods are contemplated for the quantification of PI3K activity which may be suitable for “high-throughput” applications, such as mass spectrometry of PIP3 levels in membranes, as well as ELISA-based methods, which do not require radiolabeling and thin layer chromatography (e.g. from Echolon Biosciences). PI3K activity can serve as a reliable biomarker for FXS and a potential read-out for drug screens. For quantification of protein kinase activities by LC-MS see Alcolea & Cutillas Methods Mol. Biol. 2010; 658:325-37. For a site-specific, multiplexed kinase activity assay using stable-isotope dilution and high-resolution mass spectrometry see Yu et al., Proc Natl Acad Sci USA. 2009 Jul. 14; 106(28):11606-11.

In certain embodiments, it is contemplated that an immunosorbent separation medium is used, e.g., a sandwich ELISA to detect PIP3, PI3K, or p110beta subunit. For example, in such an assay a solid substrate is conjugated with a PI3K or p110beta capture antibody; a sample is added, and PI3K or p110beta binds to the capture antibody; a second PI3K or p110beta detecting antibody is added, and binds to a second epitope/antigen on PI3K or p110beta; a third enzyme-linked or marker antibody is added, and binds to detecting antibody. After washings markers are detected or the enzyme substrate is added and converted by the enzyme to a detectable form.

In certain embodiments it is contemplated that an immunosorbent separation medium is used in a competitive ELISA to detect PIP3, PI3K or p110beta subunit. For example in such an assay a first antibody with an epitope of PIP3, PI3K, or p110beta is incubated in the presence of a sample for detection of PIP3, PI3K or p110beta subunit. Sample is added to an antigen coated well, and the well is washed, so that any unbound antibody is removed. Labeled or marker antigens are added to the wells. The more antigen, e.g., PIP3, PI3K, or p110beta, in the sample, the less labeled antibody will be able to bind to the antigen in the well, hence “competition.” A secondary antibody, specific to the first antibody is added. This second antibody is coupled to a marker or an enzyme that can make a substrate change color. In the case of an enzyme, a substrate is added, and enzymes elicit a chromogenic or fluorescent signal. For competitive ELISA, the higher the sample antigen concentration, the weaker the eventual signal.

### Terms

Currently, health care providers typically think of autism as a “spectrum” disorder, a group of disorders with similar features. One person may have mild symptoms, while another may have serious symptoms. But they both have an autism spectrum disorder. The term “autism spectrum disorder” is intended to include, but not limited to, classical autism, Asperger syndrome, pervasive developmental disorder, atypical autism, childhood disintegrative disorder, and Rett syndrome. The main signs and symptoms of autism spectrum disorders involve problems in communication, e.g., both verbal (spoken) and non-verbal (unspoken, such as pointing, eye contact, and smiling), social situations, e.g., sharing emotions, understanding how others think and feel, and holding a conversation, and stereotyped behaviors, e.g., repeating words or actions, obsessively following routines or schedules, and playing in repetitive ways.

The term “PI3K” refers to phosphoinositide 3-kinases (PI3Ks) which phosphorylate the 3-prime OH position of the inositol ring of inositol lipids. They have been implicated as participants in signaling pathways regulating cell growth by virtue of their activation in response to various mitogenic stimuli. PI3Ks are composed of a 110-kD catalytic subunit and an 85-kD adaptor subunit. The human PI3-kinase p110beta subunit has the following polypeptide sequence (SEQ ID NO: 15, MCFSFIMPPA MADILDIWAV DSQIASDGSI PVDFLLPTGI YIQLEVPREA TISYIKQMLW 61 KQVHNYPMFN LLMDIDSYMF ACVNQTAVYE ELEDETRRLC DVRPFLPVLK LVTRSCDPGE 121 KLDSKIGVLI GKGLHEFDSL KDPEVNEFRR KMRKFSEEKI LSLVGLSWMD WLKQTYPPEH 181 EPSIPENLED KLYGGKLIVA VHFENCQDVF SFQVSPNMNP IKVNELAIQK RLTIHGKEDE 241 VSPYDYVLQV SGRVEYVFGD HPLIQFQYIR NCVMNRALPH FILVECCKIK KMYEQEMIAI 301 EAAINRNSSN LPLPLPPKKT RIISHVWENN NPFQIVLVKG NKLNTEETVK VHVRAGLFHG 361 TELLCKTIVS SEVSGKNDHI WNEPLEFDIN ICDLPRMARL CFAVYAVLDK VKTKKSTKTI 421 NPSKYQTIRK AGKVHYPVAW VNTMVFDFKG QLRTGDIILH SWSSFPDELE EMLNPMGTVQ 481 TNPYTENATA LHVKFPENKK QPYYYPPFDK IIEKAAEIAS SDSANVSSRG GKKFLPVLKE 541 ILDRDPLSQL CENEMDLIWT LRQDCREIFP QSLPKLLLSI KWNKLEDVAQ LQALLQIWPK 601 LPPREALELL DFNYPDQYVR EYAVGCLRQM SDEELSQYLL QLVQVLKYEP FLDCALSRFL 661 LERALGNRRI GQFLFWHLRS EVHIPAVSVQ FGVILEAYCR GSVGHMKVLS KQVEALNKLK 721 TLNSLIKLNA VKLNRAKGKE AMHTCLKQSA YREALSDLQS PLNPCVILSE LYVEKCKYMD 781 SKMKPLWLVY NNKVFGEDSV GVIFKNGDDL RQDMLTLQML RLMDLLWKEA GLDLRMLPYG 841 CLATGDRSGL IEVVSTSETI ADIQLNSSNV AAAAAFNKDA LLNWLKEYNS GDDLDRAIEE 901 FTLSCAGYCV ASYVLGIGDR HSDNIMVKKT GQLFHIDFGH ILGNFKSKFG IKRERVPFIL 961 TYDFIHVIQQ GKTGNTEKFG RFRQCCEDAY LILRRHGNLF ITLFALMLTA GLPELTSVKD 1021 IQYLKDSLAL GKSEEEALKQ FKQKFDEALR ESWTTKVNWM AHTVRKDYRS).

As used herein a “sample” refers to a composition taken from or originating from a subject. Examples of samples include cell samples, blood samples, tissue samples, hair samples, and urine or excrement samples.

A “subject” refers to any animal such as a human patient, livestock or a domestic pet.

As used herein, the terms “prevent” and “preventing” include the prevention of the recurrence, spread or onset. It is not intended that the present invention be limited to complete prevention. In some embodiments, the onset is delayed, or the severity is reduced.

As used herein, the terms “treat” and “treating” are not limited to the case where the subject (e.g. patient) is cured and the disease is eradicated. Rather, embodiments of the present invention also contemplate treatment that merely reduces symptoms, and/or delays disease progression.

The term “antibody,” as used herein, refers to an immunoglobulin molecule which specifically binds to the epitope of an antigen. Antibodies can be intact immunoglobulins derived from natural sources or from recombinant sources and can be immunereactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

A “separation medium” refers to a stationary phase, gel, or adsorbent. In certain embodiments, the invention relates to analysis of samples using chromatographic processes or gel electrophoresis. Electrophoresis is a procedure which enables the sorting of molecules based on size and charge. An electromotive force (EMF) is used to move the molecules through the gel. The gel is typically a crosslinked polymer. When separating proteins or nucleic acids (DNA, RNA, or oligonucleotides) the gel is usually composed agarose or acrylamide, and a cross-linker. Proteins are usually denatured in the presence of a detergent such as sodium dodecyl sulfate/sodium dodecyl phosphate (SDS/SDP) that coats the proteins with a negative charge. Proteins may be analyzed by sodium dodecyl sulfate polyacrylamide gel electrophoresis (SDS-PAGE), by native gel electrophoresis, by quantitative preparative native continuous polyacrylamide gel electrophoresis (QPNC-PAGE), or by 2-D electrophoresis.

“Chromatography” refers to processes used to purify individual components from mixtures by passing a mixture contained in a “mobile phase” through a “stationary phase,” which separates the analyte to be measured from other components in the mixture. Ion exchange chromatography, liquid chromatography, normal-phase (NP) and reversed-phase chromatography (RP), affinity chromatography, and expanded bed adsorption (EBA) chromatograph all use separation mediums. In ion exchange chromatography, the separation medium is typically an ion exchange resin that carries charged functional groups which interact with oppositely charged groups of the compound to be retained. In affinity chromatography, the separation medium is typically a gel matrix, often of agarose, typically coupled with metals or molecules that bind with markers or tags such antigens, antibodies, enzymes, substrates, receptors, and ligands. Methods utilizing antibodies or antigens (epitopes) coupled to the separation medium is typically referred to as immunoaffinity chromatography and the separation medium is referred to as an immunosorbent.

Liquid chromatography (LC) is a separation technique in which the mobile phase is a liquid. Typical separation mediums for liquid column chromatography include silica gel, alumina, and cellulose powder. Liquid chromatography can be carried out under a relatively high pressure is referred to as high performance liquid chromatography (HPLC). HPLC is historically divided into two different sub-classes based on the polarity of the mobile and stationary phases. The technique in which the stationary phase is more polar than the mobile phase (e.g. toluene as the mobile phase, silica as the stationary phase) is called normal phase liquid chromatography (NPLC) and the opposite (e.g. water-methanol mixture as the mobile phase and C18=octadecylsilyl as the stationary phase) is called reversed phase liquid chromatography (RPLC).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the term “marker” is used broadly to encompass a variety of types of molecules which are detectable through spectral properties (e.g. fluorescent markers or “fluorophores”) or through functional properties (e.g. affinity markers). A representative affinity marker includes biotin, which is a ligand for avidin and streptavidin. An epitope marker or “epitope tag” is a marker functioning as a binding site for antibody. Since chimeric receptor proteins and antibodies can be produced by recombinant methods. Receptor ligands are effective affinity markers.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

### EXPERIMENTAL

### Example 1

### Excess PI3K Activity at Fmr1 KO Synapses

The molecular mechanisms underlying exaggerated receptor-mediated signaling and protein synthesis in the absence of FMRP are unknown. Some reports have revealed defects in PI3K/mammalian target of rapamycin (mTOR) signaling, whereas others implicate impaired ERK signaling in FMRP-deficient cells. Both pathways are crucial to regulate mGluR-induced protein synthesis in normal brain, yet it remains controversial whether one pathway is predominantly dysregulated in FXS. However, no study has directly examined or compared the activity of PI3K and ERK in FXS. The activity was quantified for these two downstream signaling molecules at synapses from Fmr1 KO mice. PI3K assays were performed with p110beta, the predominant neuronal isoform of PI3K catalytic subunits, immunoprecipitated from Fmr1 KO and WT SNS. The efficiency of the precipitated p110beta protein to convert phosphoinositol (PI) into phosphoinositol-3-phosphate (PI3-P) was assessed by using radiolabeled ATP to quantify PI3-P production on thin-layer chromatographs. PI3K activity in Fmr1 KO SNS is markedly increased (threefold) compared with WT (FIG. 1A, n=8, * p=0.035, paired t test). In contrast, phospho-ERK1/2 immunoprecipitated from Fmr1 KO SNS displayed no difference in the efficiency to phosphorylate recombinant ELK-1 compared with WT SNS (FIG. 1B, n=8, p=0.20, paired t test). To confirm these initial findings, we further examined PI3K activity at synapses in cultured WT and Fmr1 KO hippocampal neurons. Accumulation of the PI3K products PI-(3,4)-bisphosphate (PIP2) and PI-(3,4,5)-triphosphate (PIP3) leads to recruitment of PH-domain-containing proteins to sites of PI3K enzymatic activity (Hawkins et al., 2006), e.g., to synaptic membranes. As an in vivo measure to assess PI3K activity at synapses, we analyzed the subcellular distribution of a recombinant PH domain of murine Akt1 fused to red fluorescent protein [RFP-PH(Akt)] in hippocampal WT and Fmr1 KO neurons. In Fmr1 KO neurons, colocalization of RFP-PH(Akt) with the synaptic marker synaptophysin was significantly increased, suggesting elevated synaptic PIP3 levels as a consequence of exaggerated PI3K activity at synapses (FIG. 1C, n=5 independent experiments, 6-12 cells each, ***p=0.00002, paired t test). Total RFP-PH(Akt) levels were not changed in Fmr1 KO neurons compared to WT. Excess synaptic PI3K activity was further corroborated by significantly increased phosphorylation of the downstream signaling molecule Akt in SNS from Fmr1 KO mice (FIG. 1D, n=6, * p=0.014, paired t test), whereas ERK phosphorylation was not altered (FIG. 1E, n=7, p=0.18, paired t test). These results indicate that exaggerated gp1 mGluR signaling in FXS does not lead to excess signaling of downstream pathways in general. In contrast, the key downstream signaling molecule PI3K, but not ERK1/2, is specifically overactive at FMRP-deficient synapses.

### Example 2

### Dysregulated mGluR-Mediated PI3K Activity in Fmr1 KO Neurons

Antagonists of gp1 mGluR signaling can rescue FXS-associated neuronal phenotypes in animal models. To assess whether gp1 mGluR signaling can regulate excessive PI3K activity at FMRP-deficient synapses, the effects of different perturbations of mGluR signaling were investigated; each showed reduction of PI3K activity in Fmr1 KO. Antagonizing mGluR5-mediated signaling with MPEP had no significant effect on WT but significantly reduced PI3K function in Fmr1 KO (FIG. 2A, n=4, two-way ANOVA with least significant difference (LSD) post hoc tests: pwtctr-wtMPEP=0.327, * pwtctr-koctr=0.033, * pkoctr-koMPEP=0.012), indicating that inhibition of gp1 mGluRs can correct excessive PI3K activity in the absence of FMRP, which is consistent with the mGluR theory. Synaptic mGluR-dependent induction of PI3K can be regulated by different mechanisms, such as signaling through heterotrimeric G-proteins or a neuronal complex including mGluR5, the scaffolding protein Homer, and the PI3K enhancer PIKE-L. To investigate whether excess PI3K activity in Fmr1 KO SNS is regulated by the mGluR-Homer-PIKE signaling complex, two different approaches to interfere with this complex were used: perturbation of mGluR5-Homer interactions with a peptide comprising the C-terminal sequence of mGluR5 (FIG. 2B) and short-hairpin RNA-mediated knockdown of PIKE-L in cultured cortical neurons (FIG. 2C). Perturbation of mGluR5-Homer associations reduced PI3K activity under basal conditions independently of genotype, although PI3K activity was still increased in Fmr1 KO compared with WT SNS. Quantification of PI3K assays demonstrated a significant effect of the C-terminal peptide, which reduced PI3K activity in both WT and Fmr1 KO (FIG. 2B). In line with previous results (FIG. 1A,F), a significant effect of genotype was also observed, with increased PI3K activity in Fmr1 KO SNS compared with WT regardless of the presence of the C-terminal peptide [FIG. 2B, n=4, two-way ANOVA, significant effects of treatment (*p<0.001) and genotype (*p=0.014) but no significant interaction between genotype and treatment (p=0.143)]. Of interest, quantification of shRNA-mediated PIKE reduction showed a significant interaction between genotype and treatment to reduce the excess PI3K activity observed in Fmr1 KO. In PIKE-depleted Fmr1 KO neurons, there was a significant decrease in PI3K activity (54.5±9%) but only a slight decrease in PIKE-depleted WT neurons (93±3%) [FIG. 2C, n=3, two-way ANOVA with Tukey's honestly significant difference (HSD) post hoc tests: *pwtctr-koctr=0.002, * pkoctr-koPIKE=0.007]. However, PI3K activity is still increased in Fmr1 KO compared with WT after knockdown of PIKE (139±14% of WT), suggesting an additional downstream defect leading to excess PI3K activity in Fmr1 KO.

Although it is not intended that embodiments of the invention be limited by any particular mechanism, it is believed that excess PI3K activity in Fmr1 KO is attributable to elevated translation of FMRP-associated mRNAs that encode PI3K subunits or modulating proteins. Of interest, PIKE was identified as an FMRP target mRNA in a high-throughput screen, and quantitative FMRP-specific coimmunoprecipitation from brain lysates data suggests that PIKE-L mRNA specifically associates with FMRP. Another study reported elevated levels of PIKE-S (short isoform of PIKE) in hippocampal lysates from Fmr1 KO neurons and also suggested increased levels of both PIKE-S and PIKE-L in postsynaptic densities from Fmr1 KO (Sharma et al., (2010) J Neurosci 30:694-702.). In line with this observation, here PIKE-L protein levels are shown to significantly enriched in SNS from Fmr1 KO. Because PI3K activity is exaggerated in FMRP-deficient non-neuronal cells, which express neither gp1 mGluRs nor PIKE-L, the results imply that excess PI3K activity in Fmr1 KO might be attributed to downstream defects within the pathway.

gp1 mGluR-mediated activation of synaptic translation was shown to be occluded in Fmr1 KO (Muddashetty et al., 2007). Because PI3K is important for mGluR induction of protein synthesis, whether mGluR-induced PI3K signaling was also dysregulated in the absence of FMRP was investigated. We analyzed PI3K activity after stimulation of gp1 mGluRs with DHPG in WT and Fmr1 KO SNS. DHPG treatment led to a twofold increase of PI3K enzymatic activity in WT SNS (FIG. 2D, n=4, * p=0.033, paired t test) but failed to activate PI3K in KO SNS (FIG. 2E). Based on the finding that PI3K activity is also increased in the absence of gp1 mGluRs, this suggests that excess synaptic PI3K activity in Fmr1 KO might not be further inducible by gp1 mGluR stimulation and thus might underlie the loss of gp1 mGluR-induced protein synthesis. This finding is consistent with a previous report showing that DHPG does not induce the phosphorylation of PDK1, which is also induced by PI3K, in hippocampal slices from Fmr1 KO mice (Ronesi J A, Huber K M (2008a) J Neurosci 28:543-547). Of interest, we observe a significant reduction of PI3K activity in Fmr1 KO SNS during DHPG stimulation (FIG. 2E, n=4, * p=0.049, paired t test). The significant reduction in PI3K activity observed in Fmr1 KO SNS could be attributable to agonist-mediated mGluR5 desensitization, which was shown to decrease PI3K activity. Collectively, our findings indicate that mGluR-mediated control of PI3K activity is dysregulated in the absence of FMRP

### Example 3

### Increased p110beta Protein Levels at Fmr1 KO Synapses

p110beta is the catalytic subunit of PI3K and a putative FMRP-target mRNA. Whether p110beta expression was dysregulated in Fmr1 KO, which could contribute to the excess PI3K activity, was investigated. Synaptic p110beta protein levels in cultured hippocampal neurons were analyzed by quantifying p110beta colocalization with the synaptic marker synaptophysin (FIG. 3A-E, specificity of the antibody was verified by siRNA-mediated knockdown of FMRP in HEK293T cells. In both WT and Fmr1 KO neurons, p110beta showed a punctate distribution within dendrites and colocalized with synaptophysin, which is consistent with its known recruitment to active signaling complexes at synapses (FIG. 3A, B). Quantitative analyses of distal dendrites (>50 μm from cell body) demonstrated significantly increased synaptic localization of p110beta protein in Fmr1 KO neurons (FIG. 3C, WT, 27% overlap; KO, 34.9% overlap; n=43 dendrites each for WT and KO, 3 independent hippocampal cultures, **p=0.008, independent t test), whereas total synaptophysin levels were unchanged (FIG. 3D, n=43, p=0.73, independent t test). Furthermore, the Mander's coefficient for p110beta but not synaptophysin is significantly higher in Fmr1 KO dendrites, indicating that the percentage of p110beta signal overlapping with synaptophysin is increased in the absence of FMRP. Using three-dimensional reconstruction, p110beta protein levels within synaptophysin punctae, here referred to as “synapses” were analyzed. Although the mean intensity of p110beta within synapses was increased (FIG. 3E, n=43, ** p=0.004, independent t test), the relative number of p110beta-positive synapses was unchanged, implying that there is no imbalance between synapses recruiting PI3K versus synapses not recruiting PI3K in Fmr1 KO neurons. Additional quantitative analyses of immunostainings in hippocampal neurons showed that total dendritic p110beta levels are also increased in the absence of FMRP.

### Example 4

### Enhanced Basal p110Beta Expression and mRNA Translation in the Absence of FMRP

Western blot analyses of cortical SNS corroborated the above immunocytochemical data by showing that p110beta levels were significantly increased in Fmr1 KO (FIG. 3F, n=5, * p=0.042, paired t test). This is in line with a previous report that showed increased p110beta levels in total hippocampal lysates from Fmr1 KO mice but did not analyze synaptic levels (Sharma et al., 2010). Apart from increased synaptic levels in Fmr1 KO mice, increased p110beta protein levels in HEK293T cells was detected after siRNA-mediated knockdown of FMRP (FIG. 3G, n=5, * p=0.005, paired t test), further suggesting that gp1 mGluRs are not necessary for excess p110beta expression in the absence of FMRP.

A report suggested that p110beta mRNA might be a target mRNA of FMRP, although this could not be validated using specific in vitro assays (Miyashiro et al., (2003) Neuron 37:417-431). That FMRP associates with p110beta mRNA in vivo was demonstrated using FMRP-specific coimmunoprecipitation from brain lysate followed by qRT-PCR analyses (FIG. l H, n=6, two-way ANOVA with Tukey's HSD post hoc analyses: *pp 110beta=0.003, pNR1=0.884). Furthermore, RNA pulldowns in HEK293T cells using recombinant p110beta mRNA and FMRP demonstrated that the 3′ UTR of p110beta is sufficient to mediate the association of p110beta mRNA with FMRP in vitro (FIG. 31, n=4, two-way ANOVA with Tukey's HSD post hoc analyses: *pp110beta=0.001, pβ-actin=0.798).

The role of FMRP in p110beta mRNA translation has not been studied previously. To investigate whether p110beta translation was regulated by FMRP, translation levels were quantified in SNS by analyzing p110beta mRNA distribution in polysomal fractions of sucrose gradients. The association of p110beta mRNA with synaptoneurosomal polysomes is sensitive to puromycin treatment, which disrupts translocating ribosomes, indicating that it is actively translated (FIG. 3J, n=3, * p=0.033, paired t test). In Fmr1 KO SNS, the association of p110beta mRNA with heavy polysomes is significantly increased compared with WT (FIG. 3K, n=5, * pPSD95=0.029, pNR1=0.89, * p110beta=0.043, paired t tests), suggesting enhanced basal translation of p110beta mRNA in the absence of FMRP. In contrast, total p110beta mRNA levels were not altered in Fmr1 KO cortical lysates. Apart from these results in mouse cortical synaptoneurosomes, qRT-PCR analyses of polysomal fractions from HEK293T cells after siRNA-mediated reduction of FMRP also demonstrate that association of p110beta mRNA with puromycin-sensitive polysomes is significantly elevated (FIG. 3L, two-way ANOVA with Tukey's HSD post hoc analyses: *pctruntr-ctrpuro=0.002, * pctruntr-KDuntr=0.001, * pKDuntr-KDpuro<0.001). Our findings in HEK293T cells support our hypothesis that gp1 mGluRs or other neuron-specific receptors might not be driving the dysregulated p110beta mRNA translation and protein expression in FMRP-deficient cells. Of note, the effects on p110beta protein levels and translation are larger after transient FMRP reduction in HEK293T cells than in the Fmr1 KO SNS (protein levels: 2.5-fold increase in cells vs 20% increase in SNS; polysomal RNA: 39% increase in cells vs 28% in SNS). These results suggest that excessive translation of the PI3K catalytic subunit p110beta in the absence of FMRP leads to increased p110beta protein levels at synapses and could therefore contribute to aberrant and dysregulated synaptic PI3K activity. Fluorescent in situ hybridization analyses in cortical and hippocampal mouse brain sections also showed that p110beta mRNA can be localized to dendrites in WT and Fmr1 KO, consistent with our observations of FMRP-mediated translational regulation of p110beta mRNA in synaptic fractions.

### Example 5

### gp1 mGluR-Dependent Increase of p110beta Protein Expression is Occluded at Fmr1 KO Synapses

Stimulation of gp1 mGluRs leads to activation of PI3K signaling at synapses by recruiting catalytic p110beta molecules into receptor protein complexes at the membrane. FMRP has been shown to play an important role for the gp1 mGluR-induced protein synthesis (Muddashetty et al., 2007). Besides recruitment of existing p110beta protein into receptor protein complexes, whether gp1 mGluR signaling could additionally lead to increased p110beta mRNA translation, protein expression, and subsequently elevated p110beta protein levels at synapses was analyzed. Treatment of WT SNS with the gp1 mGluR agonist DHPG for 15 min led to increased p110beta protein levels (FIG. 4A, n=6, * p=0.008, paired t test) and induced a significant shift of p110beta mRNA into puromycin-sensitive fractions (FIG. 4B, n=6, * p=0.028, paired t test). In contrast, in Fmr1 KO SNS, gp1 mGluR-mediated increase in p110beta protein expression (FIG. 4C) or translation (FIG. 4D) were occluded (C: n=5, p=0.613; D: n=3, p=0.21; paired t tests). The loss of sensitivity of p110beta mRNA translation and protein expression to gp1 mGluR stimulation in the absence of FMRP parallels the loss of PI3K activation during gp1 mGluR stimulation observed in Fmr1 KO SNS (FIG. 2E). We thus suggest that excess p110beta mRNA translation, protein synthesis, and consequently activity under basal conditions in Fmr1 KO might occlude gp1 mGluR-induced PI3K activation that is necessary to stimulate protein synthesis.

### Example 6

### PI3K Inhibitors Rescue Increased and Dysregulated Protein Synthesis at Fmr1 KO Synapses

A prominent phenotype of Fmr1 KO mice is excess and dysregulated protein synthesis, which is hypothesized to underlie many neuronal defects observed in FXS. PI3K activates mTOR and cap-dependent translation (Costa-Mattioli et al., (2009) Neuron 61:10-26.) and is therefore an upstream regulator of protein synthesis. To examine whether excessive PI3K signaling might cause dysregulated translation in Fmr1 KO, metabolic radiolabeling was used in cortical SNS to quantify the effects of different signal transduction agonists on translation rates. A significant increase of overall synaptic protein synthesis rates was observed in Fmr1 KO SNS compared with WT (FIG. 5A, n=6, * p=0.03, paired t test (Wien et al., (2007) Neuron 56:955-962). Preincubation of Fmr1 KO SNS with inhibitors of either mGluR5 (MPEP) (FIG. 5B) or mGluR1 (LY367385) (FIG. 5C) reduced synaptic Fmr1 KO translation to

WT levels, indicating that increased translation rates were modulated by gp1 mGluR-dependent signaling pathways (FIG. 5B,C, two-way ANOVAs; B, MPEP, n=8, * pwtuntr-kountr=0.043, * pkountr-koMPEP=0.002; C, LY367385, n=5, * pwtuntr-kountr=0.04, * pkountr-koLY367=0.007; Tukey's HSD post hoc tests). This is in line with results showing that antagonizing gp1 mGluR signaling significantly reduces activity of PI3K, a crucial regulator of protein synthesis, in Fmr1 KO SNS (FIG. 2A-C). In contrast, an NMDA receptor-specific antagonist (APV), failed to reduce augmented translation in KO (FIG. 5D, n=3, two-way-ANOVA: no significant interaction between genotype and treatment, p=0.782), further corroborating that excessive synaptic translation in Fmr1 KO is attributable to exaggerated signaling downstream of gp1 mGluRs. Similar to the effect of gp1 mGluR antagonists (FIG. 5B,C), antagonizing PI3K signaling with two different drugs, LY294002 (FIG. 5E) and wortmannin (FIG. 5F), could specifically rescue the excessive synaptic translation in Fmr1 KO (FIG. 5E, LY294002, n=6, * pwtuntr-kountr=0.001, * pkountr-koLY294=0.026; FIG. 5F, wortmannin, n=6, * pwtuntr-kountr<0.001, * pkountr-kowort=0.002; two-way ANOVAs with Tukey's HSD post hoc tests), whereas an inactive analog of LY294002 (LY303511) did not show any effect. In contrast, a specific antagonist of ERK1/2 signaling (U0126) reduced protein synthesis levels independently of genotype to the same extent [FIG. 5G, n=5, two-way ANOVA: significant effects of treatment (*p=0.001) and genotype (*p=0.001) but no significant interaction between genotype and treatment (p=0.68)], and ratios of KO to WT translation rates were the same before and after U0126 treatment.

Together, these results suggest that excess PI3K activity causes excess basal translation in FXS and might therefore underlie the dysregulated basal and stimulus-induced protein synthesis in the absence of FMRP. Furthermore, treatment with rapamycin, which inhibits the PI3K downstream signaling molecule mTOR, significantly reduced translation in KO but not in WT, supporting the hypothesis that PI3K signaling is enhanced in the absence of FMRP. This result is also in line with a study reporting increased phosphorylation and activity of mTOR in the absence of FMRP (Sharma et al., 2010). Interestingly, this study showed that gp1 mGluR-induced mTOR phosphorylation, which is absent in Fmr1 KO, could be restored with a PI3K inhibitor. Here, the PI3K antagonist wortmannin was shown to rescue the translational response of Fmr1 KO SNS to gp1 mGluR stimulation, whereas treatment with an ERK1/2 antagonist did not rescue DHPG-stimulated translation in the absence of FMRP (FIG. 5H; wortmannin: n=4, one-way ANOVA, *p=0.012, Tukey's post hoc tests: *puntrctr-wortctr=0.045, * pwortctr-wortDHPG=0.010; U0126: n=4, one-way ANOVA, *p=0.001, Tukey's post hoc tests: *puntrctr-U0126ctr/DHPG=0.006/0.001, * puntrDHPG-U0126ctr/DHPG=0.019/0.004). In WT, inhibitors of either ERK1/2 or PI3K abolished DHPG-induced translational increase (FIG. 5I; wortmannin: n=4, one-way ANOVA, *p=0.002, Tukey's post hoc tests: puntrctr-untrDHPG=0.006, * pwortctr-wortDHPG=0.004; U0126: n=3, one-way ANOVA, *p<0.001, Tukey's post hoc tests: *puntrctr-untrDHPG=0.029, * puntrctr/DHPG-U0126DHPG=0.022/0.001). These data further corroborate that both ERK and PI3K/mTOR pathways are important to activate translation in normal brain, yet impaired signaling through PI3K/mTOR underlies the dysregulated translation in FXS.

### Example 7

### PI3K inhibition Corrects Aberrant AMPA Receptor Internalization and Increased Protrusion Density in Fmr1 KO Neurons

The observation that PI3K inhibitors, but not ERK1/2 inhibitors, could rescue dysregulated protein synthesis, a fundamental phenotype in FXS believed to underlie neuronal deficits observed in animal models as well as in patients, led to further investigation of the therapeutic potential of PI3K antagonists for FXS-associated defects. The effect of the PI3K antagonist LY294002 was analyzed on two important synaptic phenotypes in Fmr1 KO neurons: (1) aberrant AMPA receptor internalization and (2) increased protrusion density in Fmr1 KO dendrites.

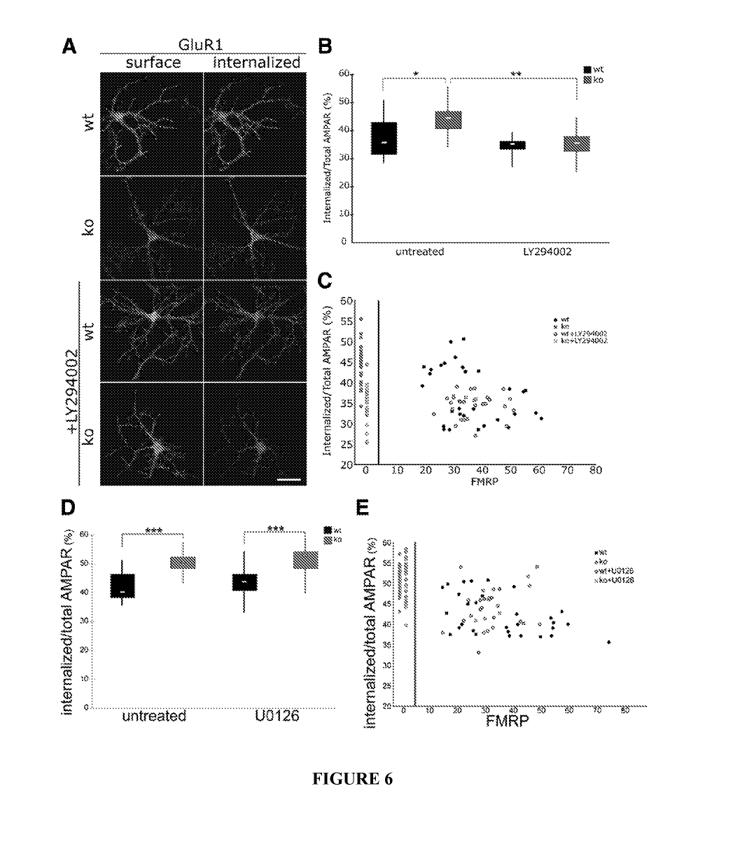

siRNA-mediated FMRP deficiency was shown to leads to aberrant internalization of AMPA receptors in rat hippocampal neurons and that the mGluR5 antagonist MPEP rescued increased levels of internalized AMPARs (Nakamoto et al., (2007) Proc Natl Acad Sci USA 104:15537-155342). Here, reduction of excess PI3K activity could also correct this phenotype was investigated. In line with observations in the rat hippocampal neuron knockdown assay, Fmr1 KO mouse hippocampal neurons similarly display excessive constitutive AMPAR internalization compared with WT neurons (FIG. 6A, B). Treatment with the PI3K inhibitor LY294002 (50 μM, 1 h) reduced AMPAR endocytosis in Fmr1 KO neurons to WT levels (FIG. 6A-C, n=30, Bonferroni's t tests, *pwtctr-koctr<0.0001, ** pkoctr-KO LY294002<0.00001). Notably, in WT neurons, FMRP expression levels in dendrites correlated with AMPAR internalization, and antagonizing PI3K had an effect on AMPAR surface expression only in neurons with low dendritic expression of FMRP, i.e., high levels of internalized AMPAR (FIG. 6C). As observed for the rescue of excess and dysregulated translation (see above), the effect was specific for PI3K antagonists, whereas the ERK1/2 inhibitor U0126 (20 μM, 1 h) did not rescue aberrant AMPAR internalization in Fmr1 KO neurons (FIG. 6D, E, n=30, Bonferroni's t test, ***p<0.0001).

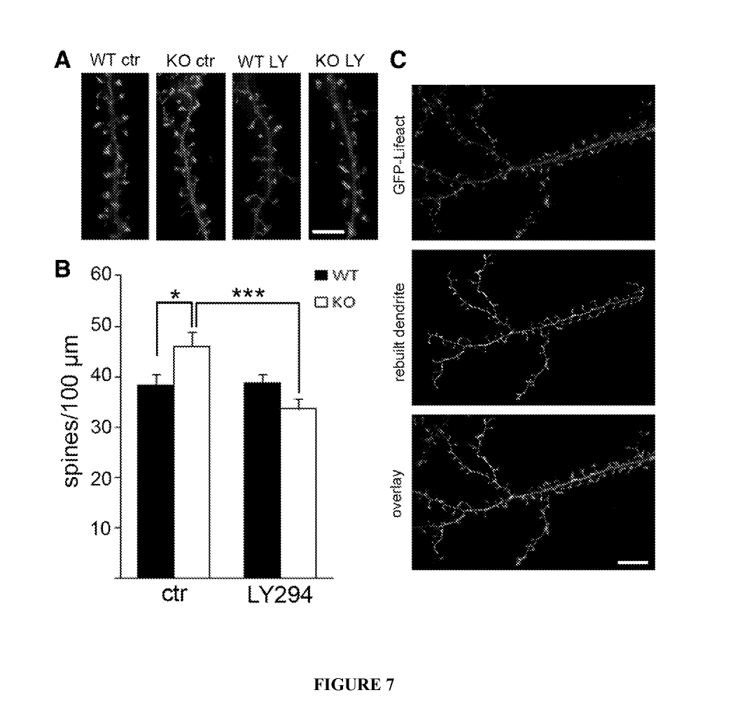

A prominent feature of FMRP-deficient neurons in humans and mice is altered dendritic spine morphology, including higher density, and immature appearance of spines, which can be corrected by genetic or pharmacologic reduction of mGluR5 signaling. Using automated software (FilamentTracer in Imaris; Bitplane), the overall protrusion density in WT and Fmr1 KO hippocampal neurons were analyzed in vitro after a 3 d treatment with the PI3K inhibitor LY294002 (10 μM, 3d) (FIG. 7A-C). To assess a potential value for a therapeutic strategy in FXS patients, a prolonged incubation with low doses of the inhibitor, as opposed to higher doses of shorter duration, was used to minimize effects on WT morphology, as well as to reduce possible side effects on neuronal function in WT and KO neurons. To visualize spines, cells were transfected with GFP-Lifeact, a small peptide that binds F-actin without altering actin morphology or dynamics. A significant increase in dendritic protrusions (including spines and filopodia) was detected in Fmr1 KO neurons, and antagonizing PI3K signaling with LY294002 could rescue the elevated protrusion density observed in Fmr1 KO neurons (FIG. 7B). In contrast, LY294002 had no effect on WT neurons (FIG. 7B, n=30, LSD post hoc tests, *pwtctr-koctr=0.024, *** pkoctr-koLY294<0.0001, pwtctr-wtLY294=0.996). These results indicate that exaggerated PI3K activity at synapses may underlie gp1 mGluR-dependent deficits in synapse morphology in the absence of FMRP.

### Methods

Compounds, siRNA, Primers, and Peptides.

Puromycin was obtained from Sigma-Aldrich; other drugs were purchased from Tocris Bioscience. Radiolabeled amino acids and trinucleotides were purchased from GE Healthcare and PerkinElmer Life and Analytical Sciences. L-Phosphatidylinositol (liver, bovine; sodium salt) was obtained from Avanti Polar Lipids. Other chemicals were purchased from Sigma-Aldrich. Tat-mGluR5-CT or tat-mGluR5-MUT peptides were synthesized by Invitrogen. Stealth small interfering RNA (siRNA) (Invitrogen) was designed using BLOCK-iT RNAi Designer (Invitrogen) (SEQ ID NO:1, for sifmr1: NM 008031 stealth 298, sense, UGG CGC UUU CUA CAA GGC AUU UGU A; SEQ ID NO:2, sictr: NM 008031 stealth control 298, sense, UGG UUU CCA UCG GAA UUA CUG CGU A). siRNA targeting p110beta was purchased from Santa Cruz Biotechnology (sc-29447, PI 3-kinase p110beta siRNA (m) is a pool of 3 target-specific 19-25 nt siRNAs).

### Primers for Quantitative Real-Time PCR.

Sequences of primers for quantitative real-time (qRT)-PCR were as follows: p110beta forward, SEQ ID NO: 3,5′-TTCTTTTCAGTGTTGTGACCAAG-3′ and reverse, SEQ ID NO: 4,5′-GCCCCGAATGTGGTAAGTT-3; NR1 forward, SEQ ID. NO: 5,5′-GGCAGTAAACCAGGCCAATA-3′ and reverse, SEQ ID NO:6,5′-AGCAGAGCCGTCACATTCTT-3; postsynaptic density protein 95 (PSD95) forward, SEQ ID NO:7,5′-CTATGAGACGGTGACGCAGA-3′ and reverse, SEQ ID NO:8,5′-CGGGAGGAGACAAAGTGGTA-3; enhanced green fluorescent protein (EGFP) forward, SEQ ID NO:9,5′-AAGGACGACGGCAACTACAAG-3′ and reverse, SEQ ID NO: 10, 5′-ATGCCGTTCTTCTGCTTGTCG-3; phosphoinositide 3-kinase enhancer, long isoform (PIKE-L) forward, SEQ ID NO: 11, 5′-TGTCCACACCATCTAAGACTGAA-3′ and reverse, SEQ ID NO: 12, 5′-AGGATTTTAGTTTCCACATTTTGC-3; and β-actin forward, SEQ ID NO:13, 5′-GCACTCTTCCAGCCTTCC-3′ and reverse, SEQ ID NO:14, 5′-CCGCTCAGGAGCAAT-3′.

### Antibodies.

Mouse monoclonal anti-tubulin, anti-PSD95, anti-GFAP, anti-MAP2, and anti-synaptophysin antibodies, as well as rabbit polyclonal anti-mGluR5 antibodies, were purchased from Millipore Corporation; mouse monoclonal anti-mGluR1 antibody was purchased from BD Biosciences. Rabbit polyclonal antibody against p110beta was purchased from Santa Cruz Biotechnology. Rabbit monoclonal antibodies specific for phosphor-Akt(Thr308), Akt(pan), and ERK1/2, as well as mouse monoclonal anti-phospho-ERK1/2(Thr202/Tyr204) antibody, were purchased from Cell Signaling Technology. Anti-PIKE-L antibody was described previously (Tang et al., 2008). Mouse monoclonal anti-FMRP antibody 7G1-1 was obtained from Developmental Studies Hybridoma Bank (University of Iowa, Iowa City, Iowa). Mouse monoclonal anti-FMRP antibody 2F5-1 (used for Western blot analyses) was a kind gift from Jennifer C. Darnell (The Rockefeller University, New York, N.Y.). A rabbit polyclonal antibody raised against the C terminus of FMRP was used for Western blot analysis of polysomal gradients.

cDNA Constructs and Plasmids

Murine flag-mCherry-FMRP (open reading frame) was subcloned from EGFP-FMRP (Antar et al., 2005), and murine EGFP-p110beta-3′ untranslated region (UTR) (GenBank accession number NM 029094.2; nucleotides 3401-4649) and murine EGFP-β-actin-3′ UTR (GenBank accession number NM 007393.3; nucleotides 1208-1860) were generated by PCR from mouse cDNA and subcloned into pEGFP-C1 (Clontech). The cDNA encoding the Pleckstrin homology (PH) domain of mouse Akt1 was generated by PCR from mouse cDNA (GenBank accession number NM 009652.2; nucleotides 285-725) and cloned into a plasmid containing cDNA encoding monomeric red fluorescent protein (RFP). GFP-Lifeact was a kind gift from Roland Wedlich-Soeldner (Max Planck Institute for Biochemistry, Martinsried, Germany).

### Kinase Activity Assays.

In vitro PI3K assays were performed as described previously (Ye et al., (2000) Cell 103:919-930) using p110beta antibody. In vitro extracellular signal-regulated kinase (ERK) activity assays were performed using the p44/42 MAP Kinase Assay kit from Cell Signaling Technology according to the manual. HEK293T cells were cultured in DMEM supplemented with 10% FBS. Fmr1-specific siRNA was transfected using LF2000 (Invitrogen) according to the manual. At 48 h after transfection, cells were scraped from the dish with PBS, divided into two equal parts for PI3K and ERK activity assays, respectively. For treatment with PI3K antagonist, cells were incubated with 40 μM LY294002 [2-(4-morpholinyl)-8-phenyl-1(4H)-benzopyran-4-one] or LY303511 (2-(1-piperazinyl)-8-phenyl-4H-1-benzopyran-4-one, inactive analog) for 15 min before further processing for PI3K assay. Cortical synaptoneurosomes (SNS) were prepared as described previously (Muddashetty et al., (2007) J Neurosci 27:5338-5348.). SNS were split in two equal parts for PI3K and ERK activity assays. Cells and SNS were lysed in kinase assay buffers (for PI3K assay: 50 mM Tris-HCl, pH 7.4, 40 mM NaCl, 1 mM EDTA, 0.5% Triton X-100, 1.5 mM Na₃VO4, 50 mM NaF, 10 mM sodium pyrophosphate, and 10 mM sodium β-glycerol phosphate, supplemented with proteinase inhibitors; for ERK activity assay: lysis buffer provided by Cell Signaling Technology). Lysates were cleared by centrifugation, and protein concentrations were determined by the Bradford method. For both kinase assays, the same amount of protein was used for wild-type (WT) and Fmr1 KO SNS or sifmr1 knockdown (KD) and sictr HEK293T cells, respectively. For measuring PI3K activity following various treatments, SNS were incubated for 10 min at 37° C. with either 100 μM (S)-3,5-dihydroxyphenylglycine (DHPG) or 10 μM 2-methyl-6-(phenylethynyl)-pyridine (MPEP) or for 1 h at 37° C. with tat-mGluR5-CT or tat-mGluR5-MUT peptides (5 μM) before lysis. For PI3K assays with cortical neurons, cells at 11d in vitro (DIV) were transfected with 10 μl of control adenovirus or adenovirus carrying short-hairpin PIKE-L (as described previously by Tang et al., (2008) Nat Cell Biol 10:698-706.) for 48 h. PI3K assays were performed with 300 μg of protein as described above. To confirm PIKE-L knockdown, 80 μg of protein was used for Western blot analyses using anti-PIKE-L antibody.

### Quantification of Western Blots.

SDS-PAGE and Western blots were performed as described previously (Muddashetty et al., 2007) and quantified using NIH ImageJ. Signal intensities of p110beta- and FMRP-specific bands were normalized to tubulin signal on the same blot. Signal intensities of phospho-proteins were normalized to total levels of the respective protein on the same blot. For this purpose, Western blots were stripped after detection with the phospho-specific antibodies using 100 mM β-mercaptoethanol and 2% SDS in 62.5 mM Tris-HCl, pH 6.8, at 50° C. for 30 min and, after extensive washing, detected with the respective antibody specific for the total protein.

### Cell Culture.

Hippocampal primary neurons were dissected from WT and Fmr1 KO mice at embryonic day 17 and cultured as described previously (Muddashetty et al., 2007). Neurons were transfected with RFP-PH(Akt) at 9 DIV using Lipofectamine 2000 (LF2000; Invitrogen) according to the manual. Six hours after transfection, neurons were fixed with 4% paraformaldehyde (PFA) and further processed for immunocytochemistry.

### Immunohistochemistry.

For p110-beta-specific staining, neurons were fixed at 17 DIV. Cells were permeabilized with 0.3% Triton X-100 and blocked with 2% normal donkey serum and 4% BSA. Primary antibodies were incubated at room temperature for 1 h (anti-p110beta at 1:50; anti-synaptophysin at 1:300) and detected with cyanine dye 3-coupled donkey anti-rabbit and cyanine dye 2-coupled donkey anti-mouse antibodies, respectively.

### Imaging and Processing.

Images of immunocytochemistry on cultured neurons were acquired using a wide-field fluorescent Nikon Eclipse inverted microscope with cooled CCD camera and built-in Z-drives. Stacks were deconvolved with AutoQuant X (Media Cybernetics). For quantification of signal intensities, as well as colocalization analyses, a region of interest along a dendrite at least 50 μm from the cell body was chosen. Mean fluorescent intensities were determined using IPlab software (BD Biosciences) or Imaris software (Bitplane) and normalized to background staining of the same area on the coverslip. Colocalization analyses were performed using Imaris Coloc software (Bitplane). Images of fluorescent in situ hybridization experiments on tissue were acquired using a Carl Zeiss LSM510 confocal microscope, deconvolved with AutoQuant X (Media Cybernetics), and are displayed as flattened z-stacks or as single slices.

### RNA Coimmunoprecipitation.

Lysates for RNA coimmunoprecipitations were prepared by homogenizing cortices from mice (C57BL/6 and Fmr1 KO, backcrossed in C57BL/6J, postnatal day 17-21) in lysis buffer (20 mM Tris-HCl, pH 7.4, 150 mM NaCl, 5 mM MgCl2, and 1% NP-40, supplemented with proteinase and RNase inhibitors) on ice. RNA coimmunoprecipitations and subsequent qRT-PCR were performed as described previously (Muddashetty et al., 2007) using the mouse monoclonal 7G1-1 antibody.

### Anti-Flag Pulldown.

HEK293T cells were transfected with flag-mCherry-FMRP and EGFP-p110beta-3′ UTR or EGFP-β-actin-3′ UTR using Lipofectamine 2000 (Invitrogen) according to the manual. For controls, EGFP constructs were cotransfected with flag-mCherry. At 24 h after transfection, cells were lysed with coimmunoprecipitation lysis buffer (20 mM Tris-HCl, pH 7.4, 150 mM NaCl, 5 mM MgCl2, and 1% NP-40, supplemented with proteinase and RNase inhibitors) on ice. Anti-flag pulldowns were performed with anti-flag M2 affinity gel (Sigma) and analyzed with EGFP-specific qRT-PCRs as described previously (Muddashetty et al., 2007).

### Linear Sucrose Gradients.

Gradient preparation, centrifugation, fractionation, RNA purification, and qRT-PCRs were performed as described previously (Muddashetty et al., 2007).

### Synaptoneurosome Preparation, Treatment, and Metabolic Labeling.

Cortical SNS were prepared as described previously (Muddashetty et al., 2007) and treated with the indicated antagonists or solvent for 5 min at 37° C., for some experiments followed by DHPG (100 μM) for 15 min at 37° C. as indicated, and with puromycin (2 mM) for 30 min at 37° C. Metabolic labeling was performed with 100 μCi Pro-Mix, L-(35S) in vitro cell labeling mix (GE Healthcare) for 5 min. Triplicate samples from time point 0 and 5 min were washed once in homogenization buffer, followed by lysis (100 mM Tris-HCl, pH 7.4, 150 mM NaCl, 0.5% deoxycholate, and 1% Triton X-100, supplemented with proteinase inhibitors) and TCA precipitation. 35S-incorporation levels were estimated by scintillation counting.

### Constitutive AMPAR Internalization Assay.

The assay was performed as described previously for rat neurons (Nakamoto et al., 2007), with the following modifications: mouse WT or Fmr1 KO cells were preincubated with LY294002 (50 μM) or U0126 [1,4-diamino-2,3-dicyano-1,4-bis(o-aminophenylmercapto)butadiene] (20 μM) for 1 h when indicated. Surface AMPARs in live neurons were labeled with a rabbit polyclonal antibody against the N terminus of the GluR1 subunit (Calbiochem) (1:5 in conditioned media) for 15 min at 37° C., 0.5% CO2. After incubation with antagonists [1 μM TTX, 10 μM NBQX (2,3-dihydroxy-6-nitro-7-sulfonyl-benzo[f]quinoxaline), and 50 μM APV (Tocris Bioscience), as described previously (Bhattacharyya et al., 2009)] in conditioned media for 15 min at 37° C., 0.5% CO2, cells were fixed in 4% PFA and processed and analyzed.

### Analysis of Dendritic Protrusion Density.

High-density hippocampal neurons from WT and Fmr1 KO were treated for 3 d with 10 μM LY294002 or an equal amount of vehicle (DMSO) starting at 15 DIV. Every 24 h, culture media was exchanged with fresh drug in conditioned media. After 48 h (17 DIV), cells were transfected (NeuroMag, according to the manual; OZBiociences) with GFP-Lifeact, a peptide that binds to filamentous actin (F-actin) and can be used to visualize dendrites and spines. At 16-24 h after transfection, cells were fixed and imaged with a Nikon Eclipse inverted microscope using a 60× Plan-Neofluar objective and a cooled CCD camera (Quantix; Photometrics). Images were taken as z-stacks (31 stacks, 0.2 μm per stack) and deconvolved using AutoQuant 2× (Media Cybernetics). Dendrites and spines (>40 μm distance from cell body, the analyzed region including a main dendrite of at least 50 μm length) were traced and analyzed using FilamentTracer (Imaris software; Bitplane). Total protrusion density was displayed as average number per 100 μm.

1. A method of treating or preventing a disease related to fragile X mental retardation protein (FMRP) comprising administering a PI3K antagonist to a subject at risk of, exhibiting symptoms of , or diagnosed with the disease.

2. A method of treating synapse defects in the brain comprising administering a PI3K antagonist to a subject.

3. The method of claim 1, wherein the subject is diagnosed with fragile X syndrome, autism, or an autism spectrum disorder.

4. The method of claim 1, wherein the PI3K antagonist is a broad spectrum PI3K antagonist.

5. The method of claim 1, wherein the PI3K antagonist preferentially binds p110beta.

6. The method of claim 1, wherein the PI3K antagonist is siNA of PI3K.

7. The method of claim 1, wherein the PI3K antagonist is siRNA of p110beta.

8. The method of claim 1, wherein the PI3K antagonist is an antibody to PI3K.

9. The method of claim 1, wherein the PI3K antagonist is an antibody to p110beta.

10. The method of claim 1, wherein the PI3K antagonist is an aptamer to PI3K.

11. The method of claim 1, wherein the PI3K antagonist is an aptamer to p110beta.

12. The method of claim 1, wherein the PI3K antagonist is wortmannin, 2-morpholin-4-yl-8-phenylchromen-4-one, 4-(2-(1H-indazol-4-yl)-6-(4-(methylsulfonyl)piperazin-1-yl)methyl)thieno[3,2-d]pyrimidin-4-yl)morpholine, N-(7,8-dimethoxy-2,3-dihydroimidazo[1,2-c]quinazolin-5-yl)nicotinamide, (R)-2-(1-(7-methyl-2-morpholino-4-oxo-4H-pyrido[1,2-a]pyrimidin-9-yl)ethylamino)benzoic acid, acetic acid (1S,4E,10R,11R,13S,14R)-[4-diallylaminomethylene-6-hydroxy-1-methoxymethyl-10,13-dimethyl-3,7,17-trioxo-1,3,4,7,10,11,12,13,14,15,16,17-dodecahydro-2-oxa-cyclopenta[α]phenanthren-11-yl ester, N-(3-(benzo[c][1,2,5]thiadiazol-5-ylamino)quinoxalin-2-yl)-4-methylbenzenesulfonamide, 24(6-amino-9H-purin-9-yl)methyl)-5-methyl-3-(o-tolyl)quinazolin-4(3H)-one, 2-methyl-2-(4-(3-methyl-2-oxo-8-(quinolin-3-yl)-2,3-dihydro-1H-imidazo[4,5-c]quinolin-1-yl)phenyl)propanenitrile, 7-methyl-2-(4-morpholinyl)-9-[1-(phenylamino)ethyl]-4H-pyrido[1, 2-a]pyrimidin-4-one, 3-[4-(4-morpholinyl)pyrido[3′,′:4,5]furo[3,2-d]pyrimidin-2-yl]-phenol, 5-[5-(4-fluoro-2-hydroxy-phenyl)-furan-2-ylmethylene]-thiazolidine-2,4-dione, 5-(6-quinoxalinylmethylene)-2,4-thiazolidinedione, 5-[(2,2-difluoro-1,3-benzodioxol-5-yl)methylene]-2,4-thiazolidinedione, (Z)-5-((4-(pyridin-4-yl)quinolin-6-yl)methylene)thiazolidine-2,4-dione, 3,3′-(2,4-diaminopteridine-6,7-diyl)diphenol or salt or prodrug thereof.

13. The method of claim 12 wherein the prodrug is RGDS-conjugated prodrug of morpholin-4-yl-8-phenylchromen-4-one.

14. A method of diagnosing a disease related to fragile X mental retardation protein (FMRP) comprising assaying a sample from a subject for excessive PI3K or PI3K activity and correlating excessive PI3K or PI3K activity to a disease related to FMRP.

15. The method of claim 14, wherein the disease is fragile X, autism, or an autism spectrum disorder.

16. The method of claim 14, wherein assaying comprises detecting PI3K, p85 or p110 subunits and comparing the detected amount to that typically found in a sample from a person with or without a disease related to fragile X mental retardation protein.

17. The method of claim 16, wherein PI3K, p85 or p110 subunits are detected by mass spectroscopy.

18. The method of claim 15, wherein assaying comprising detecting phosphatidylinositol 3-phosphate in the sample and comparing the detected amount to that typically found in a sample from a person with or without a disease related to fragile X mental retardation protein.

19. The method of claim 14, wherein the phosphatidylinositol 3-phosphate is detected by mass spectroscopy.

20. The method of claim 14, wherein the assaying comprises isolating PI3K, p85 or p110 subunits from the sample providing isolates and measuring PI3K activity in the isolates.

21. The method of claim 14, wherein the assaying comprises, combining the sample and affinity markers for PI3K, p85 or p110 subunits and measuring markers in the marker bound sample.

22. The method of claim 21, wherein the markers are antibodies for PI3K, p85, or p110 subunit.

23. The method of claim 22, wherein the markers are fluorescent.

24. The method of claim 14, wherein the assaying comprises the step of detecting expression of mRNA encoding PI3K, p85, or p110 subunit in the sample.

25. The method of claim 14, wherein the assaying comprises, mixing the sample with a polynucleotide that hybridizes to mRNA encoding PI3K, p85, or p110 subunit.

26. The method of claim 25, wherein the polynucleotide is conjugated to a fluorescent marker.

27. The method of claim 29, wherein assaying comprises moving the sample through separation medium and detecting PI3K, p85, or p110 subunit, phosphatidylinositol 3-phosphate, mRNA encoding PI3K, p85, or p110, or PI3K activity.

28. The method of claim 14, wherein the sample comprises a lymphocyte or fibroblast.
